(19) United States
(12) Patent Application Publication
(10) Pub. No.: US 2010/0130439 A1
(43) Pub. Date: May 27, 2010

(54) GENOMIC MUTATION INHIBITORS THAT INHIBIT Y FAMILY DNA POLYMERASES

(76) Inventors: Floyd E. Romesberg, La Jolla, CA (US); Daniel A. Bachovchin, San Diego, CA (US)

Correspondence Address: QUINE INTELLECTUAL PROPERTY LAW GROUP, P.C., P O BOX 458, ALAMEDA, CA, 94501, US

(21) Appl. No.: 12/451,146

(22) PCT Filed: Apr. 25, 2008
(86) PCT No.: PCT/US2008/005401

§ 371 Date: Jan. 21, 2010

Related U.S. Application Data
(60) Provisional application No. 60/926,563, filed on Apr. 26, 2007.

(51) Int. Cl. A61K 31/7072; C12Q 1/68; C40B 30/04

(52) U.S. Cl. 514/50; 435/6; 506/9

(57) ABSTRACT

Modulators of error prone DNA polymerases are provided. Methods of inhibiting genomic mutation to inhibit the emergence of drug resistant target cells are also provided. Screening assays for identifying modulators of error prone DNA polymerases are provided.

### CROSS-REFERENCE(S) TO RELATED APPLICATION(S)

This application claims priority to and benefit of U.S. Provisional Patent Application U.S. Ser. No. 60/926,563 filed Apr. 26, 2007, by Romesberg and Bachovshin entitled “Genomic Mutation Inhibitors that Inhibit Y Family DNA Polymerases” under 35 U.S.C. §119(e). This provisional application is incorporated herein by reference in its entirety.

### FIELD OF THE INVENTION

The invention is in the field of drug resistance. Modulators of DNA polymerases are used to inhibit the emergence of drug resistant cells, particularly in patients most at risk for developing drug resistance.

### BACKGROUND OF THE INVENTION

Drug resistance occurs in a variety of clinical settings and is a significant threat to both human and veterinary patients, world-wide. Drug resistance can include the evolution of resistance to chemotherapeutics in cancer or other target cells, e.g., during treatment of chronic conditions such as cancer, cystic fibrosis (CF) and arthritis, as well as the often rapid evolution of pathogen (e.g., bacterial, fungal, protozoan, etc.) resistance in a patient, e.g., during treatment.

Antibiotics, for example, have relatively short product life-spans, as resistant strains of pathogenic bacteria typically emerge, often world-wide, within a few years of drug introduction (e.g., Avom et al. (2001) World Health Organization Alliance for the Prudent Use of Antibiotics: Synthesis of Recommendations by Expert Policy Groups. Geneva: World Health Organization 155p.). Similarly, drug resistance of Plasmodium spp. is a significant problem in malaria control. Resistance has been reported to most anti-malaria drugs except (so far) artemisinin and its derivatives. Drug resistance also occurs during cancer therapy, where it is believed to be the main cause of therapeutic failure for cancers that are initially sensitive to a chemotherapeutic (Allen et al. (2002) Cancer Research 62:2294-2299).

Certain broad spectrum antibiotics, such as quinolones (e.g., ciprofloxacin), can induce relatively rapid resistance during treatment, with resistant bacteria evolving over even a relatively short treatment cycle in a particular patient. This is particularly problematic where, e.g., broad spectrum quinolone antibiotics such as ciprofloxacin are given to treat chronic infections (which can require longer and repeated treatment cycles) such as urinary tract infections (UTIs). Antibiotic treatment of certain types of infections can lead to the emergence of resistant bacteria in a given patient for other classes of antibiotics as well, even when currently effective antibiotics are used in the treatment. For example, rifamycins, which are used to treat mycobacterial infections such as tuberculosis and leprosy, can become ineffective in a patient, e.g., during the course of treatment.

The emergence of resistance is exacerbated in patients that display a predisposition to particular infections (e.g., UTIs) or diseases (e.g., cancer or CF), or that are old, immune compromised, or generally unhealthy. Further, patients that are exposed (e.g., therapeutically) to mutagens such as chemotherapeutics, radiation, UV, etc., e.g., during treatment, are also at an increased risk for the development of drug target resistant cells, because these mutagens can induce resistance in the target cells, e.g., through genomic mutation of the cells. Patients suffering from chronic conditions such as certain infections (e.g., UTIs, malaria, tuberculosis, etc.), cancer, and other chronic diseases are also particularly at an increased risk that resistance will develop during treatment.

The mechanisms by which drug resistance occurs have been the subject of recent study. For example, chromosomal genomic mutation during therapy has been shown to result in the rapid acquisition of resistance to quinolone and rifamycin antibiotics (Cirz et al. (2005) “Inhibition of Mutation and Combating the Evolution of Antibiotic Resistance,” PLoS Biology 3(6):1024-1033. Pol II, as well as the Y family of error prone DNA polymerases (e.g., Pol IV and PolV) have been implicated in the induction of resistance conferring mutations (Cirz et al., id; Boshoff et al. (2003) “DnaE2 Polymerase Contributes to the In Vivo Survival and the Emergence of Drug Resistance in Mycobacterium tuberculosis.” Cell 113:183-193). The molecular mechanism of action for the emergence of ciprofloxacin resistance has been hypothesized to include DNA repair pathways, e.g., Lexo A cleavage of RecBC-mediated repair of DNA damage, resulting in the derepression of SOS-regulated Pol II, Pol IV and Pol V polymerases (Circ et al. (2005), id.).

It would be highly desirable to identify modulators that, e.g., reduce the rate of bacterial resistance induced by antibiotic treatment, and/or that reduce the rate of drug resistance induced by other chemotherapeutics, e.g., as used in treating cancer, CF, etc. Preventing the emergence of resistance over the course of treatment would provide increased efficacy during treatment, particularly for chronic conditions, and would also extend, e.g., antibiotic product life cycles by reducing the rate at which resistant strains emerge in patient populations. The present invention provides these and other features that will be apparent upon a complete review of the following.

### SUMMARY OF THE INVENTION

The invention provides modulators of error prone DNA polymerases (e.g., Y family DNA polymerases) that modulate rates of genomic mutation in target cells. The ability to modulate the rate of genomic mutation in such cells affects the rate at which the cells can develop drug resistance. Patients that are prone to develop drug resistant cell populations (e.g., during antibiotic treatment or chemotherapy) are selected for treatment with the modulators of the invention. Examples of appropriate modulators include nucleoside and nucleotide analogs, e.g., chain termination analogs, that are incorporated by the relevant error prone polymerases.

Accordingly, in a first aspect, the invention provides methods of inhibiting error prone DNA polymerase mediated mutagenesis in a target cell population in a patient. The method includes selecting a patient on the basis that the patient would benefit from reduced mutagenesis of the population; and administering a nucleoside or nucleotide analog to the patient in an amount sufficient to inhibit DNA synthesis mediated by said error prone DNA polymerase in the population, thereby inhibiting mutagenesis in the population.

Relevant cell populations include pathogen populations (bacteria, plasmodium, fungi, etc.), as well. For example, the population can include pathogenic bacteria, e.g., pathogenic gram negative bacteria such as pathogenic strains of E. coli. The cell populations can also include target patient cells such as cancer cells (e.g., tumor cells, circulating cancer cells, etc.).

The polymerase to be modulated (e.g., inhibited) can be an error prone polymerase such as a Y-family polymerase, a Pol IV polymerase, a Pol V polymerase, a DnaE2-type polymerase, or the like. For example, the error prone DNA polymerase can be encoded by umuC, umuD, or both, or a homolog thereof.

As noted, the nucleoside or nucleotide analog can be a chain terminating nucleoside or nucleotide analog such as zidovudine or stavudine. A variety of chain terminating nucleotide/nucleoside analogs are noted herein. Nucleosides are commonly administered to patients (the nucleosides are converted into nucleotides by phosphorylation in the patient or cell), but nucleotide analogs can also be administered.

A variety of factors can be used to select the patient for administration. These include determining whether the patient suffers from a chronic or acute disease or infection. Patients suffering from a chronic disease/infection are particularly suitable targets for modulator administration. Example chronic conditions include old age, cystic fibrosis, chronic urinary tract infections and many others. Patient suffering from cancer are also good targets for the modulators of the invention, in that the formation of drug resistant cancer cells during treatment is a common cause of therapeutic failure during chemotherapeutic treatments. Thus, in one aspect, identifying the patient includes determining whether the patient is receiving or will receive an anti-cancer medication; and, determining whether the population is likely to mutate to include cancer cells that are resistant to the anti-cancer medication. A patient that is receiving or that will receive the anti-cancer medication and that comprises the population is selected for administration of the nucleoside or nucleotide analog. Relevant factors in determining risk of the patient to developing drug resistance include the type of cancer to be treated, the age of the patient, the immune status of the patient, the disease status of the patient, exposure of the population of cells to a mutagen (e.g., during radiation therapy), and the like.

Similarly with respect to treatment of patients infected with a pathogenic bacteria, identifying the patient can include determining whether the patient is receiving or will receive an antibiotic; and, determining whether the population is likely to mutate to include a resistant bacteria that is resistant to the antibiotic. A patient that is receiving or that will receive the antibiotic and that comprises the population likely to mutate to include a resistant bacteria is selected for administration of the nucleoside or nucleotide analog. The population is determined to be at an increased risk for mutagenesis, e.g., based upon the type of antibiotic to be administered, the type of bacterial infection to be treated, the age of the patient, the disease status of the patient, the immune status of the patient, or exposure of the population to a mutagen.

There are a variety of mutations that are known to lead to drug resistance. For example, various resistances to antibiotic can be developed, e.g., through a GC to AT transition mutation, a TA to GC mutation, or a frameshift mutation. Exposure to mutagens such as radiation and exposure to a chemical mutagen can increase the rate of mutation, and concomitant drug resistance.

One useful feature of the invention is that the dosages of nucleoside/nucleotide analogs that can are administered are optionally lower than dosages that are currently common for such modulators in other applications. The administration of lower doses of modulators reduces any side effects of the modulator in the patient population. Thus, the anti-mutagenesis dosage can be substantially less than a dose of the modulator used to treat a cell population (certain nucleosides are currently in use as antiviral agents, and antibacterial activity for some analogs has been observed). For example, where the population is a population of bacteria, the patient can be administered a dosage of the nucleoside or nucleotide analog that is less than an IC 50 for the nucleoside or nucleotide analog against the bacteria. For example, where the population is a population of bacteria, the patient can be administered a dosage of the nucleoside or nucleotide analog that is less than ½ or even less than ¼ of an IC 50 for the nucleoside or nucleotide analog against the bacteria.

Co-administration of the modulators of the invention with current drug therapeutics such as antibiotics or anti-cancer agents is a preferred use of the administrators of the invention, to prevent the emergence of resistance to the drug during therapy. Thus, the population can be a population of bacteria and the methods of the invention can include administering an antibiotic such as ciprofloxacin or triclosan to the patient. Inhibiting mutagenesis of the population inhibits development of resistance to the antibiotic by the bacteria. As already noted, the dosage of analog can be relatively low, e.g., about ¼ to about ½ of an IC 50 for the nucleoside or nucleotide analog against the bacteria. Similarly, where the population is a population of cancer cells, the method optionally includes administering an anti-cancer chemotherapeutic to the patient, wherein inhibiting mutagenesis of the population inhibits development of resistance to the chemotherapeutic by the cancer cells. Optionally, the patient can be administered a dosage of the nucleoside or nucleotide analog that is about ½ or even about ¼ of an IC 50 for the nucleoside or nucleotide analog against the cancer cells. Optionally, any of the methods can include administering more than one nucleoside or nucleotide analog to the patient (e.g., in combination with one or more antibiotic, chemotherapeutic or other drug).

One aspect of the invention derives from the recognition that nucleotide analogs that are genomic mutation inhibitors. Thus, screens to identify nucleoside or nucleotide genomic mutation modulators (e.g., inhibitors) are a feature of the invention. In one embodiment, a method of screening a set of nucleoside or nucleotide analogs to identify whether one or more members of the set inhibits genomic mutation in a target cell population is provided. The method includes contacting the target cell population with at least one member of the set; and, determining whether the member inhibits genomic mutation in the target cell population. All of the features noted above with respect to target cell types (e.g., pathogenic cells, cancer cells, etc.) and modulators apply to this embodiment as well.

The set optionally includes a library of at least 10 different nucleoside or nucleotide analogs, e.g., a library of at least 50, 100, 500 or more different nucleoside or nucleotide analogs. Contacting the target cell population with the member typically includes incubating the cell population in media that comprises the member.

Determining whether the member inhibits genomic mutation in the target cell population can include measuring a reversion rate for one or more known mutation in one or more marker gene initially present in the population, in the presence of the member. For example, the media can include a selection agent such as an antibiotic; determining whether the member inhibits genomic mutation in the target cell population can include determining a rate at which the population develops antibiotic resistance. The methods optionally include determining a dose response curve relating concentration of the member to the reversion rate.

In one example aspect, methods of screening a set of potential Pol V-specific inhibitor compounds for Pol V-specific inhibitory activity, to identify whether one or more members of the set inhibits genomic mutation in a target cell population are provided. The methods include contacting the target cell population with at least one member of the set; determining whether the member inhibits genomic mutation in the target cell population; and, optionally, comparing a genomic mutation inhibitory activity of the member in the assay to a genomic mutation activity of a positive control Pol V inhibitor, such as a Pol V modulator provided herein, thereby determining the relative inhibitory activity of the member compared to the positive control. Types of modulators, target cells, and assay formats described for other aspects apply here as well. The set optionally comprises a library of nucleoside or non-nucleotide potential polymerase inhibitors/modulators.

Compositions are also a feature of the invention. For example, combination doses of nucleoside/nucleotides plus therapeutic drugs (antibiotics, chemotherapeutics, etc.) are a feature of the invention. For example, the invention provides a composition comprising: an antibiotic or anti-cancer drug; and, a nucleoside or nucleotide analog. The antibiotic or anti cancer drug can be present in an amount that provides a therapeutic benefit to a patient suffering from a bacterial infection or cancer, respectively, wherein the nucleoside or nucleotide analog is present in an amount that is insufficient to have therapeutic antiviral, antibiotic or anti cancer activity, but wherein the nucleoside or nucleotide analog is present in an amount that reduces a genomic mutation frequency in cancer or bacterial cells in the patient.

For example, the nucleoside or nucleotide analog is present in the composition at a dosage of the nucleoside or nucleotide analog that, over a time of administration of the composition, is less than an IC 50 for the nucleoside or nucleotide analog against a target bacteria or cancer cell. For example, the nucleoside or nucleotide analog is present in the composition at a dosage of the nucleoside or nucleotide analog that, over a time of administration of the composition, is less than ½ or even less than ¼ of an IC 50 for the nucleoside or nucleotide analog against a target bacteria or cancer cell.

Uses of the nucleoside and nucleotide analogs of the invention are also a feature of the invention. For example, the invention provides use of a nucleoside or nucleotide analog for the manufacture of a medicament to inhibit mutation in a target cell population. Similarly, the invention provides use of a nucleoside or nucleotide analog in combination with an anti-cancer chemotherapeutic or an antibiotic for the manufacture of a medicament to inhibit mutation in a target cell population and to kill or inhibit growth of the target cell population. All of the features noted above with respect to target cell populations, etc., are applicable here as well, e.g., the target cell population can be a population of bacterial or cancer cells, etc. Inhibiting mutation in the target cell population inhibits the development of drug resistance in the population.

### BRIEF DESCRIPTION OF THE FIGURES

FIG. 1 provides two histograms showing the effect of AZT on cell viability following treatment. FIG. 1A depicts both wild-type and ΔumuDC strains treated or untreated with AZT and/or UV. FIG. 1B depicts both wild-type and ΔumuDC strains treated or untreated with AZT and/or MMS.

FIG. 2, panels A-D, provide tables 1-4. FIG. 2A shows UV-induced mutagenesis is inhibited by 2.5 ng/mL AZT. Data shown is the median and standard deviation of three separate experiments. FIG. 2B shows the characterization of mutants with a) ciprofloxacin and b) triclosan. FIG. 2C depicts AZT inhibits lactose reversion in CC102. FIG. 2D shows AZT inhibits MMS-induced mutagenesis.

FIG. 3 depicts the structures and names of dNTP analogs used in Example 2. The wavy line indicates where the structure was truncated for clarity, i.e., the sugar and triphosphate moieties are not shown. The numbers 1-30 along the right side of the scheme refer to the dNTP analog present in the reactions analyzed in the corresponding lanes of FIGS. 4 and 5.

FIG. 4 depicts the results of Polη and Dpo4 screens. Reactions corresponding to 14 of the dNTPs tested are shown (selected based on preliminary data). The reaction lanes are numbered 1-15 from right to left, indicating which dNTP analog is present according to the list in FIG. 3 (i.e. lane 1 corresponds to no analog, while lane 2 corresponds to P7AI, lane 3 to IN, etc.). On the right side (for Dpo4) inhibition is evident in lanes 8 and 13 (indicated with blue arrows), and on the left side (for Polη) in lanes 6-8, 13 and 14 (indicated with blue arrows).

FIG. 5 shows a gel of Polκ and Polι screens. The reaction lanes are numbered 1 to 30 from right to left, indicating which dNTP analog is present according to the list in FIG. 3 (i.e. lane 1 corresponds to no analog, while lane 2 corresponds to P7AI, lane 3 to 1N, etc.). On the right side (for Polι), inhibition is evident in lanes 8, 13, 17 and 19 (indicated with blue arrows), and on the left side (for Polκ), in lanes 6-8 and 13 (indicated with blue arrows).

### DETAILED DESCRIPTION OF THE INVENTION

Suppressing mutation pathways in target cells, including pathogen cells (bacteria, fungi, plasmodium, etc.) and cancer cells inhibits the formation of drug resistance in patients and patient populations. See also, Cirz et al. (2005) “Inhibition of Mutation and Combating the Evolution of Antibiotic Resistance,” PLoS Biology 3(6):1024-1033; Romesberg et al. COMPOSITIONS AND METHODS TO REDUCE MUTAGENESIS WO2005/056754; and Romesberg et al. COMPOSITIONS AND METHODS FOR ENHANCING DRUG SENSITIVITY AND TREATING DRUG RESISTANT INFECTIONS AND DISEASES (All incorporated herein by reference for all purposes in their entirety). The present invention, e.g., provides specific modulators (typically inhibitors) of genomic mutagenesis.

These modulators inhibit error prone (e.g., Y family) DNA polymerases, inhibiting error prone polymerase reactions (e.g., “translesion synthesis” (TLS)) in target cells. Inhibiting error prone DNA synthesis, in turn, suppresses the rate of emergence of drug-resistant cell populations in patients and in patient populations. This is particularly useful in treating patients that are prone to the formation of drug resistant cell populations, e.g., due to the type of drug administered, the disease, infection or disorder to be treated, or to the overall health status of the patient. The modulators of the invention are useful both for increasing the efficacy of drug (e.g., antibiotic and chemotherapeutic) therapy and for increasing the lifespan of drugs that are prone to develop global resistance over time, such as antibiotics.

In addition to modulators with therapeutic benefits, the invention provides modulator screening assays and platforms for the identification of additional modulators of Y family DNA polymerases, e.g., taking example modulators and the structure of relevant polymerases into account. Further details regarding each of these features are found herein.

### DEFINITIONS

Before describing the present invention in detail, it is to be understood that this invention is not limited to particular modulators, assays or biological systems, which can, of course, vary. It is also to be understood that the terminology used herein is for the purpose of describing particular embodiments only, and is not intended to be limiting. As used in this specification and the appended claims, the singular forms “a”, “an” and “the” optionally include plural referents unless the content clearly dictates otherwise. Thus, for example, reference to a “bacteria” optionally includes cultures of bacterial cells, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

A “target cell population” is a population of cells, typically in a patient, that are a target for therapeutic treatment. Examples include pathogen cells such as infectious bacteria, fungi, parasites, plasmodium, etc., as well as tumor cells, cancer cells or other therapeutically relevant drug target cells of the patient.

A “nucleoside” is a glycosylamine in which a nucleobase (often referred to simply as a “base”) is coupled to a sugar (e.g., ribose or deoxyribose) ring. Common nucleobases include the purines (e.g., adenine and guanine) and the pyrimidines (e.g., cytosine, thymidine and uracil). Naturally occurring examples of nucleosides include cytidine, uridine, adenosine, guanosine, thymidine. Nucleosides can be phosphorylated (e.g., by kinases in a cell), typically with from 1 to three phosphates, to produce “nucleotides,” which form the basic building blocks of nucleic acids (e.g., DNA and RNA). For purposes of the invention, a “nucleoside analog” is nucleoside (or structurally related moiety) other than cytidine, uridine, adenosine, guanosine, or thymidine that can be phosphorylated into a corresponding nucleotide analog and incorporated into a nucleic acid by a DNA polymerase such as an error-prone DNA polymerase (e.g., a Y family DNA polymerase such as a Pol IV or Pol V polymerase). Useful analogs include “blocking” or “chain terminating” nucleotide analogs (and their corresponding nucleoside analogs) that are incorporated into a nucleic acid, e.g., during primer extension on a DNA template by an error-prone DNA polymerase, where incorporation of the nucleotide results in chain termination. A common example of such nucleotide analogs includes dideoxy nucleosides. The error prone DNA polymerases tend to have more room at the active site of the enzyme than typical polymerases, resulting in the ability of the polymerase to incorporate more diverse nucleotide analogs than other typical polymerases.

An “error prone DNA polymerase” is a polymerase that replicates a DNA template with relatively low fidelity. Examples include the “Y family” DNA polymerases and, to a lesser extent, the RT family DNA polymerases, as well as a few members of the B family (DNA polymerase a and C which have no proofreading activity; most other B family polymerases display high fidelity). The Y-family polymerases, for example, display low fidelity even when replicating undamaged templates; these polymerases also display a strong ability to replicate through damaged DNA templates. Because of this later property, members of this family (along with, e.g., DNA polymerase α and ζ) are also known as “translesion synthesis” (TLS) polymerases. Depending on the lesion, TLS polymerases can bypass the damage in a relatively error-free or error-prone fashion, the latter resulting in elevated mutagenesis. In E. coli, two TLS polymerases of the Y family, Pol IV (DING) and Pol V (UMUC), are known. In eukaryotes, examples include Pol η (eta), Pol ι (iota), Pol κ (kappa) and Rev1 (Y family polymerases) and Polζ (zeta) (a B family polymerase).

A Pol V DNA polymerase is a Y family polymerase that is homologous to UMUC from E. coli. A Pol IV DNA polymerase is a Y family polymerase that is homologous to DINB from E. coli.

An “IC 50” in the context of a nucleoside or nucleotide analog (or other modulator) against a bacterial or other target cell is an amount of the analog that inhibits the growth of half of an inoculum of the target cell population. This is typically estimated by doing viable cell counts with varying concentrations of the analog and generating an estimate inhibition curve of the results. See, e.g., Soothilla et al. (1992) “The IC50: an exactly defined measure of antibiotic sensitivity Journal of Antimicrobial Chemotherapy” Journal of Antimicrobial Chemotherapy 29, 137-139. The “minimum inhibitory concentration” (MIC), a related but separate metric, is the minimum concentration of agent that inhibits the growth of the cultured cell. This is usually assigned a particular value, e.g., MICs at which 50% and 90% of a culture is inhibited are referred to, e.g., as a “MIC₅₀” or a “MIC₉₀”, respectively. See also, Dorsthorst et al. (2002) “Comparison of Fractional Inhibitory Concentration Index with Response Surface Modeling for Characterization of In Vitro Interaction of Antifungals against Itraconazole-Susceptible and -Resistant Aspergillus fumigatus Isolates” Antimicrob Agents Chemother. 46(3): 702-707.

### Target Cell Populations

The methods and compositions of the invention can be directed to preventing the emergence of drug resistance for any type of target cell. Typical target cells include pathogenic microorganisms (e.g., bacteria, plasmodium, fungi, etc.) and cell populations of the patent (e.g., cancer cells). A single patient can, of course, include multiple target populations, e.g., a cancer patient can include cancer cells that are being treated with a chemotherapeutic while the patient is also infected with a pathogenic bacteria, plasmodium, fungus, or the like. In fact, as is discussed in more detail below, disease states can be interrelated, e.g., cancer, immune disorders and/or cystic fibrosis can lead to increased incidence of pathogenic organism infection. Modulators of the invention can be administered to reduce mutation rates in multiple target cell populations simultaneously, reducing the risk of resistance developing against more than one type of drug (e.g., a genomic mutation inhibitor of the invention can simultaneously reduce mutation rates in cancer cells and bacterial cells, preventing antibiotic and cancer chemotherapeutic drug resistance at the same time.

Bacteria represent one preferred class of target cells in which the modulators of the invention can reduce the rate of mutagenesis. These can be gram positive or gram negative. Example targets include E. coli (which can cause, e.g., UTI, meningitis, peritonitis, mastitis, septicemia and Gram-negative pneumonia), as well as clinically relevant mycobacteria such as the M. tuberculosis complex group, which causes tuberculosis (e.g., M. tuberculosis, M. bovis, M. africanum, and M. microti); M. leprae (which causes Hansen's disease (leprosy)); and the nontuberculous mycobacteria (NTM) that cause pulmonary disease, lymphadenitis, skin disease, disseminated diseases, etc. Other clinically relevant bacteria that represent additional preferred targets, and some of the diseases associated with these bacteria include: Vibrio cholerae (Cholera), Treponema pallidum (syphilis), Bacillus anthracis (anthrax), Yersinia pestis (bubonic plague), Streptococcus pneumoniae, Neisseria meningitidis, Pseudomonas aeruginosa, Staphylococcus aureus, Streptococcus agalactiae, Haemophilus influenzae and Listeria monocytogenes (e.g., Bacterial meningitis). Drug resistant and multi-drug resistant bacteria represent a preferred class of target cells, because increased multi-drug resistance can occur when treating a resistant or multi-resistant bacteria. Known resistant bacteria include, e.g.: ciprofloxacin-resistant S. aureus, coagulase-negative Staph, E. faecalis, E. faecium, E. coli, K. oxytoca, K. pneumoniae, M. morganii, P. mirabilis, S. marcescens, Acinetobacter, and P. aeruginosa; levofloxacin-resistant S. pneumoniae, S. pyogenes, S. agalactiae, Viridans group, E. coli, K. oxytoca, K. pneumoniae, M. morganii, P. mirabilis, S. marcenscens, Acinetobacter, and P. aeruginosa; sulfamethoxazole trimethoprim-resistant E. coli, K. oxytoca, K. pneumoniae, M. Morganii, P. mirabilis, S. marcenscens, Acinetobacter, and P. aeruginosa; ampicillin-resistant S. aureus, coagulase-negative staph, E. faecalis, E. faecium, and S. pneumoniae; oxacillin-resistant S. aureus and coagulase-negative staph; penicillin-resistant S. pneumoniae and Virdans group; piperacillin-tazobactam-resistant E. coli, K. oxytoca, K. pneumoniae, M. morganii, P. mirabilis, S. marcescens, Acinetobacter, and P. aeruginosa; cefapine-resistant S. aureus, coagulase-negative staph, S. pneumoniae, E. coli, K. oxytoca, K. pneumoniae, M. morganii, P. mirabilis, S. marcescens, Acinobacter, and P. aeruginosa; cefotaxime-resistant S. aureus, coagulase-negative staph, S. pneumoniae, E. coli, K. oxytoca, K. pneumoniae, M. morganii, P. mirabilis, S. marcenscens, Acinetobacter, and P. aeruginosa; ceftriaxone-resistant S. aureus, coagulase-negative staph, S. pneumoniae, M. morganii, P. mirabilis, S. marcescens, Acinetobacter, and P. aeruginosa; gentamycin-resistant S. aureus, coagulase-negative staph, E. faecalis, E. faecium, E. coli, K. oxytoca, K. pneumoniae, M. morganii, P. mirabilis, S. marcenscens, Acinobacter, and P. aeruginosa; clarithromycin-resistant S. pneumoniae, S. pyogenes, S. agalactiae, and Virdans group; erythromycin-resistant S. pneumoniae, S. pyogenes, and S. agalactiae, and Virdans group; teicoplanin-resistant E. faecium; vancomycin-resistant E. faecalis and E. faecium; and imipenem-resistant Acinobacter and P. aeruginosa, as well as many others known to those of skill.

As with bacterial infections, fungal infections are often chronic and/or recurring. Accordingly, treatment with a modulator of the invention can reduce the development of fungal resistance in a patient or population. Examples of fungal infections that can be treated with a modulator of the invention (i.e., in combination with an available antifungal agent) according to methods of the invention include: superficial mycoses; cutaneous mycoses (e.g., caused by dermatophytes, typically termed ringworm or tinea, e.g., caused by Microsporum, Trichophyton, or Epidermophyton); subcutaneous mycoses (chronic infections), systemic mycoses due to primary or opportunistic pathogens and others. Typical examples of fungal infections include, e.g., tinea, athlete's foot, jock itch, and candida.

Drug resistance is also a significant problem during cancer therapy, and, accordingly, cancer cells represent another preferred target cell population. Examples of cancers that may benefit from treatment with the modulators of the invention, to reduce the formation of cancer cells that are resistant to chemotherapeutics include: acute and chronic lymphocytic and granulocytic tumors, adenoma, adrenal cancer, basal cell carcinoma, bone cancer, brain cancer, breast cancer, bronchial cancer, cancer of the larynx, cancer of the nervous system, cancer of the gallbladder, cervical dysplasia and carcinoma, colon cancer, chronic myleoid lymphoma, epidermoid carcinoma, Ewing's sarcoma, hairy-cell leukemia, hyperplasia, hyperplastic corneal nerve tumor, intestinal ganglioneuromas, islet cell tumor, giant cell tumors, glioblastoma multiforme, head and neck cancers, Kaposi's sarcoma, kidney cancer, leukemias (including acute myelogenous leukemia), liver cancer, lung cancer, lymphoma, malignant carcinoid, malignant hypercalcemia, malignant melanomas, marfanoid habitus tumor, medullary carcinoma, metastatic skin carcinoma, mucosal neuromas, mycosis fungoide, myeloma, neuroblastoma, osteo sarcoma, ovarian cancer, pancreatic cancer, parathyroid cancer, primary or secondary brain tumor, pheochromocytoma, prostate cancer, rhabdomyosarcoma, rectal cancer, renal cell cancer, retinoblastoma, seminoma, soft tissue sarcoma, skin cancer, small-cell lung tumor, stomach cancer, squamous cell carcinomas of both ulcerating and papillary types, thyroid cancer, topical skin lesions, veticulum cell sarcoma, and Wilm's tumor.

Selecting Patients that Benefit from Reduced Target Cell Population Mutagenesis

A variety of patients particularly benefit from the administration of a modulator of the invention to reduce the appearance of drug resistance in the patient. Patients that benefit from modulator administration are selected, e.g., based upon the type of antibiotic to be administered, the type of bacterial infection to be treated, the age of the patient, the disease status of the patient, the immune status of the patient, exposure of the population to a mutagen, and the like.

Examples of infectious diseases that can develop resistance to drug treatment are discussed above and elsewhere herein. Table A, below, provides additional information for certain examples of infectious diseases that can develop resistance to medications during treatment and that represent targets for modulators of the present invention.

TABLE A
Examples of Infectious Diseases With Increased Resistance to Medications

| | |
|---|---|
| Urinary Tract Infection (UTIs and | Escherichia coli and |
| other chronic infections are | Staphylococcus saprophyticus. Less |
| particularly preferred targets) | commonly, Proteus mirabilis, |
| | Klebsiella pneumoniae, |
| | Enterobacter spp, and Enterococcus |
| | spp. |
| Tuberculosis | e.g., Mycobacterium tuberculosis |
| | (bacterium) |
| Cryptosporidiosis | Cryptosporidium parvum |
| | (protozoan) |
| Diphtheria | Corynebacterium diptheriae |
| | (bacterium) |
| Malaria | Plasmodium species (protozoan) |
| Meningitis, necrotizing fasciitis | Group A Streptococcus (bacterium) |
| (flesh-eating disease), toxic-shock | |
| syndrome, and other diseases | |
| Pertussis (whooping cough) | Bordetella pertussis (bacterium) |
| Schistosomiasis | Schistosoma species (helminth) |

The disease state or overall health of the patient can also be used to select patients that will most benefit from treatment with a modulator such as a nucleoside or nucleotide analog that inhibits error prone polymerases such as the Y DNA polymerases (e.g., Pol V and/or Pol IV). The disease state can be a chronic condition to be treated, such as a chronic infection (as discussed above, UTIs, tuberculosis and others provide examples of such chronic infections). The disease state can also be indirectly related to the condition to be treated, e.g., where a chronic disease such as old age, cystic fibrosis, cancer, or a disorder that compromises the immune system (e.g., age, lupus, HIV infection, or immune suppressive therapy such as occurs following an organ transplant). In the later case, the disease state being treated may simply be a bacterial infection, but the likelihood that the infection will recur (or that additional infections will occur) in the patient are higher than for patients that do not suffer from such diseases/disorders.

Accordingly, patients that suffer, e.g., from chronic conditions such as an age related disorder, cystic fibrosis (CF) or cancer (particularly prolonged/chronic forms of cancer) will particularly benefit from treatment with such modulators, e.g., during antibiotic therapy to treat opportunistic infections that occur as a result of the disease state. For example, CF is caused by a mutation in the cystic fibrosis transmembrane conductance regulator (CFTR) gene. The CFTR product helps create sweat, digestive juices, and mucus. Without this gene, CF patients are extremely susceptible to infection, with the result that CF patients undergo repeated antibiotic and often antifungal treatments. For example, in CF patients, thick mucous production and an impaired immune system cause repeated lung and other airway infections, which are treated, though often not actually cured, by oral and intravenous antibiotics and other medications. Accordingly, CF patients frequently comprise target cells (e.g., infectious bacteria) that are preferred targets for treatment in the present invention. That is, because CF is a chronic condition that requires frequent and repeated antibiotic and other drug treatments to address opportunistic pathogens, the problem of antibiotic resistance management is particularly important for these patients.

Cancer patients often have similar issues, e.g., when suffering from mesothelioma, lung or other airway cancers, etc. Cancer patients also often undergo radiation or high dose mutagen (e.g., chemotherapy) treatments. Thus, cancer patients are desirable targets for treatment with the modulators of the invention for several overlapping reasons: first, the cancer patient may develop drug resistance to a chemotherapeutic treatment agent, resulting in the development of resistant forms of cancer in the patient. In this instance, it is desirable to administer modulators of the invention during the course of chemotherapy. This is particularly true for patients suffering from chronic or prolonged forms of cancer. Second, the overall health status of the cancer patient is often impaired, leading to an increased risk of bacterial, fungal or other infection, due to suppressed immune response, increased contact with infectious patients in hospitals during repeated visits and stays in the hospital for cancer treatment, and the like. In this instance, it is desirable to co-administer the modulators of the invention with an appropriate antibiotic/antifungal or other appropriate medication to treat any opportunistic infection that arises. Third, cancer patients are often exposed therapeutically to potent mutagens such as high-dose radiation and mutagenic chemotherapeutics; modulators of the invention can be co-administered with such treatments to reduce the risk of induced mutagenesis in cancer or other target cells present in such patients.

The type of antibiotic or other drug agent to be administered is another patient selection criteria. Many drugs (including many antibiotics) are known to induce the formation of resistance even during relatively short treatment cycles. As has already been noted, for example, quinolone (e.g., fluoroquinolone) and rifamycin type antibiotics, particularly when used for treatment of chronic conditions such as CF, UTIs and tuberculosis, are known to suffer from induced resistance during extended or repeated treatment cycles. Any time that a drug type/regimen is known to have a relatively high likelihood of developing resistance during treatment, a modulator of the invention can desirably be administered. It is expected that one of skill is aware of many such drugs and regimens.

Combination Therapies

In one aspect, the invention includes the administration of drugs in combination with a modulator of the invention. Many different classes of drugs are known, including chemotherapeutics, antibiotics, and the like. For an introduction to the topic, see, Remington, The Science and Practice of Pharmacy 21^{st} Edition (2005) Lippincott Williams & Wilkins ISBN-10: 0781746736; # ISBN-13: 978-0781746731.

The modulators of the invention are, in one aspect, desirably administered to reduce bacterial mutagenesis during antibiotic treatment. Examples of antibiotics that may be coformulated or administered with a polymerase inhibitor/modulator of the invention include aminoglycosides, carbapenems, cephalosporins, cephems, glycopeptides, fluoroquinolones/quinolones, macrolides, oxazolidinones, penicillins, streptogramins, sulfonamides, and tetracyclines. While these preferred classes are discussed in some detail below for exemplary purposes, the issues surrounding antibiotic resistance are well-known, and one of skill can use a modulator of the invention similarly with any antibiotic that induces antibiotic resistance.

The rifamycins are a well-recognized group of antibiotics that are synthesized by the bacterium Amycolatopsis mediterranei, or artificially based upon natural compounds from Amycolatopsis mediterranei and derivatives thereof. Rifamycins are particularly effective against mycobacteria, which, as noted above, cause a host of chronic illnesses, such as tuberculosis, leprosy, and mycobacterium avium complex (MAC) infections. Examples of significant rifamycin antibiotics include rifamycin B, rifampicin, rifabutin, rifapentine and rifaximin. Drug resistance is particularly problematic in these antibiotics, because they are used to treat chronic infections. These include mycobacterial infections treated by rifamycins, including tuberculosis, leprosy, and mycobacterium avium complex (MAC) infections. Examples of specific modulators that have been demonstrated to reduce the induction of resistance by rifamycins are provided in the example sections herein.

Quinolone therapy represents another preferred target for modulator co-administration, due to the relatively rapid rate at which resistance appears during quinolone treatment. Resistance to quinolones can occur by one or more of at least three different mechanisms. These include chromosomal mutations in the genes encoding gyrase and/or topoisomerase II, and by bacterial porin and efflux pathway gene mutations. Enzyme mutations result, e.g., in an alteration of enzyme regions where the drug binds to the enzyme; the drug then exhibits a reduced affinity for the target site, rendering the drug inactive. Mutations can also result in changes in the porosity of outer membrane porin proteins of gram-negative bacteria, causing decreased permeability of the outer membrane towards the drug so that less drug reaches the target enzyme. Finally, mutations that enhance the bacteria's drug efflux capability increase the amount of drug pumped out of the cell. These mutations result from the selective pressure of exposure of the organism to antimicrobial agents during therapy and can cause treatment failure and the development of resistant strains of bacterial pathogens, particularly in hospital settings. This is especially true when treating UTIs or other chronic infections with quinolones, making co-administration with the modulators of the invention especially preferred during quinolone treatment of chronic infections.

In non-resistant bacteria, Quinolones inhibit bacterial DNA gyrase and/or topoisomerase II, thereby inhibiting DNA replication and transcription and killing the bacteria. Quinolones can enter non-resistant bacterial cells relatively easily; accordingly, they are often used to treat highly dangerous intracellular pathogens such as Legionella pneumophila and Mycoplasma pneumoniae. Examples of therapeutically significant quinolone antibiotics include “first generation” quinolones such as cinoxacin (Cinoxacin®), flumequine (Flubactin®) (Veterinary use), nalidixic acid (NegGam®, Wintomylon®), oxolinic acid, piromidic acid and pipemidic acid; “second generation” quinolones such as ciprofloxacin (Cipro®, Ciproxin®), enoxacin (Enroxil®, Penetrex®), fleroxacin (Megalone®), lomefloxacin (Maxaquin®), nadifloxacin, norfloxacin (Noroxin®, Quinabic®, Janacin®), ofloxacin (Floxin®, Oxaldin®, Tarivid®), pefloxacin and rufloxacin; “third generation” quinolones such as balofloxacin, grepafloxacin (Raxar®), levofloxacin (Cravit®, Levaquin®), pazufloxacin Mesilate, sparfloxacin (Zagam®), temafloxacin, tosufloxacin; and “fourth generation” quinolones such as clinafloxacin, gemifloxacin (Factive®), moxifloxacin (Avelox®), gatifloxacin (Tequin®, Zymar®), sitafloxacin, trovafloxacin (Trovan®), as well as quinolones in development such as ecinofloxacin and prulifloxacin (e.g., King et al. (2000) “New Classification and Update on the Quinolone Antibiotics” American Family Physician 61(6):2741; see also, the “Quinolone fact sheet” at cdc.gov/ncidod/dhqp/ar_lab_quinlolones.html). Nonfluorinated quinolones have recently been described (Jones et al. (2002) Antimicrob Agents Chemother. 46:1651-7. Ciprofloxacin dimers have also been described (Gould et al. (2004), Antimicrob Agents Chemother. 48:2108-15). Examples of specific modulators that have been demonstrated to reduce the induction of resistance by the quinolone ciprofloxacin are provided in the example sections herein.

As has already been noted, quinolones are broad spectrum antibiotics that are the first line of defense against highly (and rapidly) lethal infections such as meningitis, as well as against many chronic infections such as UTIs. Quinolones are also used to treat sexually transmitted diseases (e.g., gonorrhea, chlamydial urethritis/cervicitis, pelvic inflammatory disease), gram-negative gastrointestinal infections, soft tissue infections, ophthalmic infections, dermatological infections, surgical site infections, sinusitis, and respiratory tract infections (e.g., bronchitis, pneumonia, and tuberculosis). Quinolones are also commonly used in combination with other antibiotics to treat conditions, such as multi-drug resistant tuberculosis, neutropenic cancer patients with fever, and anthrax. Quinolones are also often used to treat infections of unknown origin.

In addition to improving treatment efficacy by reducing the rate at which bacteria mutate to form resistant pathogens, the product lifespan of quinolones and other antibiotics can be improved by co-administration of nucleoside/nucleotide analogs such as AZT and other mutation inhibitors of the invention. Co-administration of the modulators of the invention, in conjunction with current generation antibiotics such as currently effective quinolones reduces the formation of resistant pathogen strains, for example when treating infections by pathogens known to develop quinolone or other forms of antibiotic resistance. Resistance to various quinolones has been observed in at least: Escherichia coli, Klebsiella pneumoniae, and other enteric organisms; Pseudomonas aeruginosa; Chlamydia trachomatis and Mycoplasma pneumoniae; Campylobacter jejuni; Burkholderia cepacia; Stenotrophomonas maltophilia; Neisseria gonorrhoeae; Staphylococcus aureus (especially oxacillin-resistant strains); Enterococcus faecium; and Streptococcus pneumoniae (“Quinolone fact sheet” at cdc(dot)gov/ncidod/dhqp/ar_lab_quinlolones(dot)html).

Aminoglycosides are another group of antibiotics that benefit from co-administration of a modulator of the invention. Aminoglycosides are used against gram-negative bacteria, including for treatment of chronic infections, such as complicated urinary tract infections, as well as to treat septicemia, peritonitis and other severe intra-abdominal infections, severe pelvic inflammatory disease, endocarditis, mycobacterium infections, neonatal sepsis, and some ocular infections. They are also frequently used in combination with penicillins and cephalosporins to treat both gram-positive and gram-negative bacteria. Examples of aminoglycosides include amikacin, gentamycin, tobramycin, netromycin, streptomycin, kanamycin, paromomycin, and neomycin.

Carbapenems, like quinolones, are broad-spectrum antibiotics that are used to fight gram-positive, gram-negative, and anaerobic microorganisms. The modulators of the invention can be desirably used in conjunction with Carbapenems. Carbapenems are often used to treat serious single or mixed bacterial infections, such as lower respiratory tract infections, urinary tract infections, intra-abdominal infections, gynecological and postpartum infections, septicemia, bone and joint infections, skin and skin structure infections, and meningitis. Examples of carbapenems include imipenem/cilastatin sodium, meropenem, ertapenem, and panipenem/betamipron.

Cephalosporins and cephems are broad spectrum antibiotics used to treat gram-positive, gram-negative, and spirochaetal infections. Cephalosporins and cephems can be used to treat common urinary tract infections and upper respiratory infections (e.g., pharyngitis and tonsillitis), either of which can be chronic in nature, and particularly benefiting from co-administration with a modulator of the invention. Cephalosporins and cephems are also used to treat ear infections, skin infections, bronchitis, lower respiratory infections (pneumonia), and bone infections and are also a preferred antibiotic for surgical prophylaxis. Examples of cephalosporins include cefixime, cefpodoxime, ceftibuten, cefdinir, cefaclor, cefprozil, loracarbef, cefadroxil, cephalexin, and cephradineze. Examples of cephems include cefepime, cefpirome, cefataxidime pentahydrate, ceftazidime, ceftriaxone, ceftazidime, cefotaxime, cefteram, cefotiam, cefuroxime, cefamandole, cefuroxime axetil, cefotetan, cefazolin sodium, cefazolin, cefalexin.

Glycopeptide antibiotics are used to treat bacteria that are resistant to other antibiotics, such as methicillin-resistant staphylococcus aureus (MRSA). Co-administration of a modulator of the invention can lead to increased product life for antibiotics used to treat resistant pathogens, reducing the rate at which additional multi-drug resistant strains can evolve. These antibiotics are also used to treat patients that are allergic to penicillin. Patients that are allergic to such normally preferred antibiotics for a given treatment therapy represent a preferred target for co-administration therapy, in order to reduce the formation of pathogen resistance to such “back-up” antibiotics. They are also a preferred target because there are fewer treatments available to them if resistance does develop during treatment, and because such back up antibiotics may not work as well as the normally preferred front-line antibiotics, leading to longer treatment cycles that increase the likelihood that a resistant pathogen will develop during treatment. Examples of glycopeptides include vancomycin, teicoplanin, and daptomycin.

Macrolides, like quinolones, are broad-spectrum antibiotics and are often an alternative to penicillins or cephalosporins for treatment. Macrolides are usefully co-administered with the modulators of the invention, e.g., for the treatment of chronic infections. For example, macrolides are used to treat chronic respiratory tract infections (including otitis media, chronic sinusitis, bronchitis, pharyngitis, pneumonia, tonsillitis, and strep throat), sexually transmitted diseases (e.g., infections of the cervix and urinary tract, genital ulcer disease in men, syphilis), and opportunistic infections (e.g., pneumonia and mycobacterium avium complex (MAC) infection). Examples of macrolides include erythromycin, clarithromycin, azithromycin, axithromycin, dirithromycin, troleandomycin, oleandomycin, roxithromycin, and telithromycin.

Oxazolidinones are commonly administered to treat gram-positive infections. Carbapenems are used to treat gram-positive, gram-negative, and/or anaerobes. Oxazolidinones are commonly used as an alternative to other antibiotic classes for bacteria that have developed resistance. As has already been noted, such “back up” antibiotics are desirably co-administered with a modulator of the invention to increase the product life of the antibiotic and because there is an increased risk that resistance or multi-drug resistance can develop during treatment. Examples of oxazolidinones include linezolid.

Penicillins are broad spectrum antibiotics used to treat gram-positive, gram-negative, and spirochaetal infections. Depending on the type of infection to be treated, co-administration with a modulator can be desirable. As has been noted, treatment of chronic or repeated infections benefits from co-administration of a modulator of the invention. Conditions commonly treated with penicillins, any of which can be chronic or repeated, e.g., due to congenital or environmental factors, include meningitis, dermatological infections, ear infections, respiratory infections, urinary tract infections, acute sinusitis, pneumonia, and lyme disease. Examples of penicillins include penicillin, amoxicillin, amoxicillin-clavulanate, ampicillin, ticarcillin, piperacillin-tazobactam, carbenicillin, piperacillin, mezocillin, benzathin penicillin G, penicillin V potassium, methicillin, nafcillin, oxacillin, cloxacillin, and dicloxacillin.

Streptogramins are antibiotics used to treat drug resistant pathogens. Examples include quinupristin/dafopristin and pristinamycin. Antibiotics used to treat resistant pathogens are desirably co-administered with a modulator of the invention to increase the product life of the antibiotic and because there is an increased risk that multi-drug resistance can develop during streptogramin treatment.

Sulphonamides are broad-spectrum antibiotics that suffer from bacterial resistance, making them suitable for co-administration with the modulators of the invention. Suphonamides are commonly used to treat recurrent attacks of rheumatic fever, urinary tract infections, prevention of infections of the throat and chest, traveler's diarrhea, whooping cough, meningococcal disease, sexually transmitted diseases, toxoplasmosis, and rhinitis. Examples of sulfonamides include co-trimoxazole, sulfamethoxazole trimethoprim, sulfadiazine, sulfadoxine, and trimethoprim.

Tetracyclines are broad-spectrum antibiotics that are often used to treat gram-positive, gram-negative, and/or spirochaetal infections. Tetracyclines are often used to treat mixed infections, such as chronic bronchitis and peritonitis, urinary tract infections, rickets, chlamydia, gonorrhea, lyme disease, and periodontal disease. Tetracyclines are also used to treat chronic infections such as acne, making them suitable for co-administration with the modulators of the invention. Examples of tetracyclines include tetracycline, demeclocycline, minocycline, and doxycycline.

Similar to antibiotics, most, if not all, chemotherapeutics used for the treatment of cancer suffer from the emergence of resistant target cells, in the form of chemotherapeutic-resistant cancers, during therapy. Indeed, the development of drug-resistant cancers is considered to be the single most significant obstacle in curing cancer. This is often addressed currently by co-administering various “cocktails” of chemotherapeutics, in the hope that target cancer cells will be less likely to possess or develop resistance to more than one therapeutic. Chemotherapeutics differ from antibiotics in that most forms of cancer are not transmissible; thus, while resistant cancer cells develop in an individual, they are less likely to develop in a population than is the case for infectious pathogens such as bacteria (some cancers, e.g., those caused by viruses, are transmissible, and resistant cancers can emerge in populations of patients for these cancers). The main reason for chemotherapy failure during treatment is drug resistance, where tumors are either innately resistant to the drugs available, or else are initially sensitive but evolve resistance during treatment and eventually re-grow (Allen et al. (2002) Cancer Research 62, 2294-2299). Indeed, nearly half of all patients with cancer suffer from tumors that are intrinsically resistant to chemotherapy, and many of the remaining half develop drug resistance during the course of their treatment. Pinedo and Giaccone (eds) 1998 Drug Resistance in the Treatment of Cancer Series: Cancer: Clinical Science in Practice, Cambridge University Press ISBN-13: 9780521473217; ISBN-10: 0521473217.

A variety of chemotherapeutics are known and the likelihood that resistance will develop against given chemotherapeutics is also known. For a review of the topic, see; Di Paolo et al. (2007) “Drug distribution in tumors: mechanisms, role in drug resistance, and methods for modification” Curr Oncol Rep. 9(2):109-14; 1: Choi et al. (2006) “Platinum transporters and drug resistance” Arch Pharm Res. 12:1067-73; Chen et al. (2006) “The great multidrug-resistance paradox” ACS Chem. Biol. 1(5):271-3; Teicher (2006) Cancer Drug Resistance Series: Cancer Drug Discovery and Development ISBN: 978-1-58829-530-9; Wadhwa et al. (2002) CANCER GENE THERAPY: Scientific Basis Annual Review of Medicine 53:437-452; Gottesman et al. (2002) MECHANISMS OF CANCER DRUG RESISTANCE Annual Review of Medicine Vol. 53: 615-627; Pinedo and Giaccone (eds) 1998 (id); Pastan et al. (1991) MULTIDRUG RESISTANCE Annual Review of Medicine Vol. 42: 277-284; and the like.

Cancer drugs include (some members in the following list represent the same drug, by alternate nomenclatures): 13-cis-Retinoic Acid; 2-CdA 2-Chlorodeoxyadenosine; 5-Azacitidine 5-Fluorouracil 5-FU; 6-Mercaptopurine 6-MP 6-TG 6-Thioguanine; Abraxane, Accutane®, Actinomycin-D, Adriamycin®, Adrucil®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Ara-C, Aranesp®, Aredia®, Arimidex®, Aromasin® Arranon® Arsenic Trioxide, Asparaginase, ATRA, Avastin®, Azacitidine, BCG, BCNU, Bevacizumab, Bexarotene, BEXXAR®, Bicalutamide, BiCNU, Blenoxane®, Bleomycin, Bortezomib, Busulfan, Busulfex®, C225, Calcium Leucovorin, Campath®, Camptosar®, Camptothecin-11, Capecitabine, Carac™, Carboplatin, Carmustine, Carmustine Wafer, Casodex®, CC-5013, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine, Cytarabine Liposomal, Cytosar-U®, Cytoxan®, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin diftitox, DepoCyt™, Dexamethasone, Dexamethasone acetate, Dexamethasone Sodium Phosphate, Dexasone, Dexrazoxane, DHAD, DIC, Diodex, Docetaxel, Doxil®, Doxorubicin, Doxorubicin liposomal, Droxia™, DTIC, DTIC-Dome®, Duralone®, Efudex®, Eligard™, Ellence™, Eloxatin™, Elspar®, Emcyt®, Epirubicin, Epoetin alfa, Erbitux™, Erlotinib, Erwinia L-asparaginase, Estramustine, Ethyol, Etopophos®, Etoposide, Etoposide Phosphate, Eulexin®, Evista®, Exemestane, Fareston®, Faslodex®, Femara®, Filgrastim, Floxuridine, Fludara®, Fludarabine, Fluoroplex®, Fluorouracil, Fluorouracil (cream), Fluoxymesterone, Flutamide, Folinic Acid, FUDR®, Fulvestrant, G-CSF, Gefitinib, Gemcitabine, Gemtuzumab ozogamicin, Gemzar®, Gleevec™, Gliadel® Wafer, GM-CSF, Goserelin, Halotestin®, Herceptin®, Hexadrol, Hexylen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate Hydroxyurea, Ibritumomab, Ibritumomab Tiuxetan, Idamycin®, Idarubicin, Ifex®, IFN-alpha, Ifosfamide, IL-11, Imatinib mesylate, Imidazole Carboxamide, Interferon alfa Interferon Alfa-2b (PEG Conjugate), Interleukin ±2, Interleukin-11, Intron A® (interferon alfa-2b), Iressa®, Irinotecan, Isotretinoin, Kidrolase®, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRONT™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant, Purinethol®, Raloxifene, Revlimid®, Rheumatrex®, Rituxan®, Rituximab, Roferon-A® (Interferon Alfa-2a), Rubex®, Rubidomycin hydrochloride, Sandostatin®, Sandostatin LAR®, Sargramostim, Solu-Cortef®, Solu-Medrol®, Sorafenib, SPRYCEL™ STI-571, Streptozocin, SU11248, Sunitinib, Sutent®, Tamoxifen, Tarceva®, Targretin®, Taxol®, Taxotere®, Temodar®, Temozolomide, Teniposide, TESPA, Thalidomide, Thalomid®, TheraCys®, Thioguanine, Thioguanine Tabloid®, Thiophosphoamide Thioplex®, Thiotepa, TICE®, Toposar®, Topotecan, Toremifene, Tositumomab, Trastuzumab, Tretinoin, Trexall™, Trisenox®, TSPA TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, and Zometa®. Many of these drugs have one or more additional trade name. See also, www(dot)chemocare(dot)com/bio/. For any of these (or other known therapeutics) that suffer from the emergence of resistant cancer cells, a modulator of the invention can be added to reduce the rate that the drug induces resistance in a target cancer cell population.

It is unnecessary to discuss mechanisms of resistance for all known cancer drugs, which one of skill is generally aware of in any event. Resistance to drug treatment can emerge for essentially any chemotherapeutic known above, e.g., from Cisplatin (a platinum coordinated complex), to Trastuzumab (Herceptin). See, e.g., Choi et al. (2006) “Platinum transporters and drug resistance” Arch Pharm Res. 12:1067-73 and Dieras (2007) “Trastuzumab (Herceptin) and breast cancer: mechanisms of resistance” Bull Cancer. 94(3):259-66. For example, Cisplatin is a widely used cancer drug used for the treatment of tumors of the testis, ovarian tumors, lung cancer, advanced bladder cancer and other solid tumors. The cytotoxic action of the drug is associated with its ability to bind DNA to form cisplatin-DNA adducts. The development of resistance to cisplatin during treatment is common and constitutes a major obstacle to the cure of sensitive tumors. Cisplatin resistance occurs following mutation of relevant platinum transporters, including copper transporters (CTRs), organic cation transporters (OCTs) and multi-drug resistance related transporters (MDRs). In the case of Trastuzumab, (Herceptin), a humanized monoclonal antibody to HER2 is used to treat patients whose tumor demonstrates an amplified copy number for the HER2 oncogene and/or overexpresses the HER2 oncoprotein. Despite a high level of efficacy in combination with chemotherapy, trastuzumab as single agent has limited effectiveness (up to 30% response rates) and patients who respond to trastuzumab will relapse despite continued treatment. The mechanism of trastuzumab action is not fully understood but has been related to cell cycle inhibition. Co-expression of growth factor receptors (EGFR family, IGF-1R), and the activation of PI3K-Akt pathway, mainly by loss of PTEN function may be responsible for the resistance phenotype. The modulators of the invention can be used to suppress the emergence of resistance by blocking genomic mutation in tumor or other cancer cells, increasing the efficacy of these and many other chemotherapeutics.

### Modulators of Error Prone DNA Polymerases

DNA polymerases have relatively recently been classified into six main groups based upon various phylogenetic relationships, e.g., with E. coli Pol I (class A), E. coli Pol II (class B), E. coli Pol III (class C), Euryarchaeotic Pol II (class D), human Pol beta (class X), and E. coli UmuC/DinB and eukaryotic RAD30/xeroderma pigmentosum variant (class Y). For a review of polymerases generally, see, e.g., Hübscher et al. (2002) EUKARYOTIC DNA POLYMERASES Annual Review of Biochemistry Vol. 71: 133-163; Alba (2001) “Protein Family Review: Replicative DNA Polymerases” Genome Biology 2(1):reviews 3002.1-3002.4; Steitz (1999) “DNA polymerases: structural diversity and common mechanisms” J Biol Chem 274:17395-17398 and Burgers et al. (2001) “Eukaryotic DNA polymerases: proposal for a revised nomenclature” J Biol. Chem. 276(47):43487-90. In one preferred aspect, modulators (e.g., inhibitors) of class Y polymerases are provided in the present invention. These modulators can inhibit genomic mutation by blocking DNA damage and repair pathways that utilize class Y polymerases. Inhibiting genomic mutation reduces the rate at which target cell populations develop drug resistance traits.

Error Prone DNA Polymerases

The Y-family DNA polymerases, a preferred target for modulators of the invention, provide a superfamily of homologous proteins that exist in cells to bypass DNA damage caused by environmental damage, e.g., caused by radiation, chemicals, or other mutagens. See, Goodman (2002) “Error-prone repair DNA polymerases in prokaryotes and eukaryotes” Annu Rev Biochem. 71:17-50. For example, Escherichia coli possesses two members of the Y DNA polymerases. See, e.g., Lee et al. (2006) “Homology modeling of four Y-family, lesion-bypass DNA polymerases: the case that E. coli Pol IV and human Pol kappa are orthologs, and E. coli Pol V and human Pol eta are orthologs” J Mol Graph Model (1):87-102. These are Pol IV (dinB) and Pol V (umuD′C). In eukaryotes, polymerases η, ι, κ, and Rev1 are Y-family DNA polymerases. See also, Burgers et al. (2001). “Eukaryotic DNA polymerases: proposal for a revised nomenclature” J. Biol. Chem. 276 (47): 43487-90; and Prakash et al. (2005) “Eukaryotic translesion synthesis DNA polymerases: specificity of structure and function.” Annual Review of Biochemistry 74, 317-53. As noted above, in both prokaryotes and eukaryotes, the Y family polymerases are involved bypassing genomic DNA damage.

Polymerases that belong to the Y family are often referred to as specialized or error-prone DNA polymerases, to distinguish them from previously discovered and well-characterized DNA polymerases (e.g., Pol I, II, and DI polymerases in prokaryotes and Pol α, Pol δ, Pol ε, Pol β, Pol γ and Pol δ in eukaryotes), which display higher fidelity during template-based DNA synthesis. Pol I has also been implicated in DNA repair, having both 5′->3′(Nick translation) and 3′->5′ (Proofreading) exonuclease activity. Pol II is also involved in replication of damaged DNA having both 5′->3′ chain extension ability and 3′->5′ exonuclease activity. Pol III is the main polymerase in bacteria (it elongates copy DNA during DNA replication); it has 3′->5′ exonuclease proofreading ability. In eukaryotes, Pol a acts as a primase (synthesizing a RNA primer), and then as a DNA Polymerase, elongating the primer with (DNA) nucleotides. Elongation is taken over by Pol S and c after a few nucleotides. Pol β is implicated in repairing DNA. Pol γ replicates mitochondrial DNA. Pol 8 is the main polymerase in eukaryotes, extending primers during replication.

Y-family DNA polymerases display lower fidelity than, e.g., Pol I, II or DI (or the eukaryotic polymerases noted above), but have the capacity to replicate DNA through chemically damaged template bases, and/or to elongate mismatched primer termini. These properties occur as a result of the Y-family DNA polymerases' ability to use distorted primer templates within their active site. Y-family DNA polymerases also lack exonuclease activity, so do not have a proof-reading mechanism to correct mis-inserted bases during an extension reaction. Induction of Y-family polymerases results in an increase in the rate of genomic mutagenesis, which, in the context of the present invention, leads to increased drug resistance. Inhibiting these Y-family polymerases reduces the rate of genomic mutagenesis, reducing the rate at which drug resistance will arise in a target cell population.

For example, Pol V is the major lesion bypass polymerase involved in damage-induced mutagenesis. The role of Pol IV is less clear, but it is likely involved in error correction during SOS responses. Both dinB and umuDC loci are controlled, at least to a degree, by SOS response pathways. Cells typically have multiple Y-family DNAPs, presumably to conduct translesion synthesis (TLS) on DNA lesions of varying structure and conformation. For example, G to T mutagenesis pathways depend DNA Pol V.

The molecular mechanisms of action of the Y polymerases is beginning to be clarified. For example, Pol V is a component of a multiprotein complex that includes RecA protein, beta sliding-clamp, and single-strand DNA binding protein (SSB). See also, Paetzel et al. (2004) “UmuD and UmuD′ proteins” In: Barrett et al. eds. Handbook of Proteolytic Enzymes, 2d ed. London: Academic Press, Elsevier Science 1976-1981; and Shen et al. (2003) “Escherichia coli DNA polymerase V subunit exchange: a post-SOS mechanism to curtail error-prone DNA synthesis” J Biol Chem 278:52546-52550.

Modulators

Modulators of the invention typically inhibit Y family DNA polymerase activity. Several different types of modulators are contemplated, including nucleoside and nucleotide analog, RNAi and anti-sense modulators that inhibit Y family DNA polymerase activity, and others.

Nucleobase Y Family DNA Polymerase Inhibitors

Nucleobase modulators such as nucleoside or nucleotide analogs are preferred Y family DNA polymerase (e.g., Pol V and Pol IV) inhibitors of the invention. As noted above, error prone Y family DNA polymerases, which are induced in response to, e.g., genomic damage, mutagens, stress, etc., lack proof reading mechanisms common in other polymerases, facilitating their role in trans-lesion DNA synthesis. The lack of a proof reading mechanism provides a convenient way to specifically target the activity of the Y family polymerases, e.g., by incorporating nucleotide analogues that result in chain termination or other synthesis disruptions during DNA synthesis by the Y family polymerase.

Nucleoside analogs are typically administered to patients. However, nucleotide analogs can also be administered. Nucleosides are typically phosphorylated in the cell to become nucleotides, where they can be incorporated into DNA or other nucleic acids in the cell. A variety of nucleoside analogues are currently administered as antiviral agents, e.g., for the treatment of HIV and other viral infections. Some nucleoside analogues also have antibacterial activity per se, and have been administered as antibiotic agents. Administering such known therapeutic analogues is particularly convenient, because the toxicities and side effects for many such analogues are known. Dosages can be, e.g., similar to that used for current antiviral and antibacterial treatment regimens, but one advantage of the invention is that the anti-mutation effects of the analogues may occur at lower doses (e.g., as shown in the Examples section herein). For example, when the target cell population is a population of bacteria, the dosages of the nucleoside or nucleotide analogue can be, e.g., less than an IC 50 (or, alternately, less than the MIC₅₀) for the nucleoside or nucleotide analog against the bacteria. The dosage can thus be in the range of about ¼ to about ½ of the typical anti-viral or anti-bacterial dosage for the analogue, reducing side effects caused by administration (as compared to dosing for antiviral or antibacterial activity). Thus, administration can be, e.g., about 10%, about 20%, about 25%, about 40%, about 50%, about 75%, or about 90% or more as compared to a typical antiviral or antibiotic dose. In some embodiments, higher doses can also be administered, e.g., 100%, or more of the typical antibiotic or antiviral dose, e.g., where mutation inhibition is particularly needed, and/or where there are few side effects of the administration.

A variety of nucleoside analogues are in therapeutic use, including: AZT (zidovudine, 3′-azido-2′: deoxythymidine, Retrovir®), d4T (stavudine, 2′,3′-didehydro-3′-deoxythymidine, Zerit®, Zerit XR®), ddI (Didanosine, Videx®, Videx ECO), ddc (Zalcitabine dideoxycytidine, Hivid®). 3TC (Lamivudine Epivir®), ABC (guanosine analogue, Abacavir, Ziagen®), FTC (Emtricitabine, Emtriva®, Coviracil). Nucleotide analogues are also in use, including Tenofovir (tenofovir disoproxil fumarate, Viread®) and Adefovir (bis-POM PMPA, Preveon®, Hepsera®). These nucleoside and nucleotide analogues have similar mechanisms of action, and one of skill is aware of additional similar analogues that share the basic mechanism of action of these example analogues. In general, active analogues compete with natural deoxynucleotides for incorporation into the growing Y DNA polymerase synthesized DNA chain. However, unlike the natural deoxynucleotides substrates, these analogues can lack a 3′-hydroxyl group on the deoxyribose moiety. As a result, following incorporation of an analogue, the next incoming deoxynucleotide cannot form the next 5′-3′ phosphodiester bond needed to extend the DNA chain. Thus, these analogues are often referred to as “chain terminators.”

Non Nucleoside/Nucleotide Modulators

Non-nucleoside Y family polymerase modulators/inhibitors (NNYPIs) can also be used. A variety of non-nucleoside inhibitors of other polymerases, such as the viral reverse transcriptase enzyme, are in clinical use, including the non-nucleoside reverse transcriptase inhibitors (NNRTIs) such as Nevirapine (Viramune®), Delavirdine (Rescriptor®), Efavirenz (Sustiva®, Stocrin®). These NNRTIs may have activity as NNYPIs against the Y family polymerases. NNYPIs are not incorporated into DNA but instead typically bind to domains of the polymerase that are needed to carry out the process of DNA synthesis.

The crystal structure of various Y family polymerases is known (Ling et al. (2001) “Crystal Structure of a Y-Family DNA Polymerase in Action: A Mechanism for Error-Prone and Lesion-Bypass Replication” Cell, 107(1):91-102, and Silvian et al. (2001) “Crystal structure of a DinB family error-prone DNA polymerase from Sulfolobus solfataricus” Nat Struct Biol, 8: 984-989; see also, Perlow-Poehnelt et al. (2004) The Spacious Active Site of a Y-Family DNA Polymerase Facilitates Promiscuous Nucleotide Incorporation Opposite a Bulky Carcinogen-DNA Adduct” The Journal Of Biological Chemistry 279(35) 36951-36961. One of skill is able to design modulators that bind, e.g., the active site, or other features of the polymerase that are involved in making the polymerase active, using any available crystal structure and a molecular modeling software or system. Based on the modeling, one or more feature affecting e.g., nucleotide access to the active site and/or binding of a nucleotide within the active site region is identified, e.g., in the active site or proximal to it. Alternately, the crystal structure of any particular polymerase target can be determined, and putative modulators can be selected based on predicted binding interactions with structural features of the polymerase, e.g., binding of a putative modulator at or proximal to the active site, where the modulator can block entry of nucleotides into the active site, rendering the polymerase inactive. Ideally, such modulators are specific for the Y family polymerase target at issue, i.e., the modulator is designed to bind a structural feature of the polymerase that is unique to the Y family polymerases, e.g., that is not found in the other polymerases necessary for patient survival.

The three-dimensional structures of a large number of DNA polymerases have been determined by x-ray crystallography and nuclear magnetic resonance (NMR) spectroscopy, including the structures of polymerases with bound templates, nucleotides, and/or nucleotide analogues. Many such structures are freely available for download from the Protein Data Bank, at (www(dot)rcsb(dot)org/pdb, including structures of the Y family DNA polymerases. Structures, along with domain and homology information, are also freely available for search and download from the National Center for Biotechnology Information's Molecular Modeling DataBase, at www(dot)ncbi(dot)nlm(dot)nih(dot)gov/Structure/MMDB/mmdb(dot)shtml. The structures of additional polymerases can be modeled, for example, based on homology of the polymerases with polymerases whose structures have already been determined. Alternatively, the structure of a given polymerase, optionally complexed with a nucleotide, or the like, can be determined. In either case, the crystal structure is assessed for binding potential to libraries of putative modulator structures, thereby selecting modulators for further analysis.

Techniques for crystal structure determination are well known and can be applied to determine the structures of Y family DNA polymerases, e.g., where the structures are not already available. See, for example, McPherson (1999) Crystallization of Biological Macromolecules Cold Spring Harbor Laboratory; Bergfors (1999) Protein Crystallization International University Line; Mullin (1993) Crystallization Butterwoth-Heinemann; Stout and Jensen (1989) X-ray structure determination: a practical guide, 2nd Edition Wiley Publishers, New York; Ladd and Palmer (1993) Structure determination by X-ray crystallography, 3rd Edition Plenum Press, New York; Blundell and Johnson (1976) Protein Crystallography Academic Press, New York; Glusker and Trueblood (1985) Crystal structure analysis: A primer, 2nd Ed. Oxford University Press, New York; International Tables for Crystallography, Vol. F. Crystallography of Biological Macromolecules; McPherson (2002) Introduction to Macromolecular Crystallography Wiley-Liss; McRee and David (1999) Practical Protein Crystallography, Second Edition Academic Press; Drenth (1999) Principles of Protein X-Ray Crystallography (Springer Advanced Texts in Chemistry) Springer-Verlag; Fanchon and Hendrickson (1991) Chapter 15 of Crystallographic Computing, Volume 5 IUCr/Oxford University Press; Murthy (1996) Chapter 5 of Crystallographic Methods and Protocols Humana Press; Dauter et al. (2000) “Novel approach to phasing proteins: derivatization by short cryo-soaking with halides” Acta Cryst. D56:232-237; Dauter (2002) “New approaches to high-throughput phasing” Curr. Opin. Structural Biol. 12:674-678; Chen et al. (1991) “Crystal structure of a bovine neurophysin-II dipeptide complex at 2.8 Å determined from the single-wavelength anomalous scattering signal of an incorporated iodine atom” Proc. Natl. Acad. Sci. USA, 88:4240-4244; and Gavira et al. (2002) “Ab initio crystallographic structure determination of insulin from protein to electron density without crystal handling” Acta Cryst. D58:1147-1154.

In addition, a variety of programs to facilitate data collection, phase determination, model building and refinement, and the like are publicly available. Examples include, but are not limited to, the HKL2000 package (Otwinowski and Minor (1997) “Processing of X-ray Diffraction Data Collected in Oscillation Mode” Methods in Enzymology 276:307-326), the CCP4 package (Collaborative Computational Project (1994) “The CCP4 suite: programs for, protein crystallography” Acta Crystallogr D 50:760-763), SOLVE and RESOLVE (Terwilliger and Berendzen (1999) Acta Crystallogr D 55 (Pt 4):849-861), SHELXS and SHELXD (Schneider and Sheldrick (2002) “Substructure solution with SHELXD” Acta Crystallogr D Biol Crystallogr 58:1772-1779), Refmac5 (Murshudov et al. (1997) “Refinement of Macromolecular Structures by the Maximum-Likelihood Method” Acta Crystallogr D 53:240-255), PRODRG (van Aalten et al. (1996) “PRODRG, a program for generating molecular topologies and unique molecular descriptors from coordinates of small molecules” J Comput Aided Mol Des 10:255-262), and O (Jones et al. (1991) “Improved methods for building protein models in electron density maps and the location of errors in these models” Acta Crystallogr A 47 (Pt 2):110-119).

Techniques for structure determination by NMR spectroscopy are similarly well described in the literature. See, e.g., Cavanagh et al. (1995) Protein NMR Spectroscopy: Principles and Practice, Academic Press; Levitt (2001) Spin Dynamics: Basics of Nuclear Magnetic Resonance, John Wiley & Sons; Evans (1995) Biomolecular NMR Spectroscopy, Oxford University Press; Wüthrich (1986) NMR of Proteins and Nucleic Acids (Baker Lecture Series), Kurt Wiley-Interscience; Neuhaus and Williamson (2000) The Nuclear Overhauser Effect in Structural and Conformational Analysis, 2nd Edition, Wiley-VCH; Macomber (1998) A Complete Introduction to Modern NMR Spectroscopy, Wiley-Interscience; Downing (2004) Protein NMR Techniques (Methods in Molecular Biology), 2nd edition, Humana Press; Clore and Gronenborn (1994) NMR of Proteins (Topics in Molecular and Structural Biology), CRC Press; Reid (1997) Protein NMR Techniques, Humana Press; Krishna and Berliner (2003) Protein NMR for the Millenium (Biological Magnetic Resonance), Kluwer Academic Publishers; Kiihne and De Groot (2001) Perspectives on Solid State NMR in Biology (Focus on Structural Biology, 1), Kluwer Academic Publishers; Jones et al. (1993) Spectroscopic Methods and Analyses: NMR, Mass Spectrometry, and Related Techniques (Methods in Molecular Biology, Vol. 17), Humana Press; Goto and Kay (2000) Curr. Opin. Struct. Biol. 10:585; Gardner (1998) Annu. Rev. Biophys. Biomol. Struct. 27:357; Wüthrich (2003) Angew. Chem. Int. Ed. 42:3340; Bax (1994) Curr. Opin. Struct. Biol. 4:738; Pervushin et al. (1997) Proc. Natl. Acad. Sci. U.S.A. 94:12366; Fiaux et al. (2002) Nature 418:207; Fernandez and Wider (2003) Curr. Opin. Struct. Biol. 13:570; Ellman et al. (1992) J. Am. Chem. Soc. 114:7959; Wider (2000) BioTechniques 29:1278-1294; Pellecchia et al. (2002) Nature Rev. Drug Discov. (2002) 1:211-219; Arora and Tamm (2001) Curr. Opin. Struct. Biol. 11:540-547; Flaux et al. (2002) Nature 418:207-211; Pellecchia et al. (2001) J. Am. Chem. Soc. 123:4633-4634; and Pervushin et al. (1997) Proc. Natl. Acad. Sci. USA 94:12366-12371.

The structure of a polymerase can, as noted, be directly determined, e.g., by x-ray crystallography or NMR spectroscopy, or the structure can be modeled based on the structure of the polymerase and/or a structure of an available Y family polymerase crystal structure. The active site region of the polymerase, one preferred target for non-nucleotide modulators of the invention, can be identified, for example, by homology with other polymerases, examination of polymerase-template or polymerase-nucleotide co-complexes, and/or the like.

Such modeling of the active site to select modulators that structurally mate with features of the active site or other target of interest can involve simple visual inspection of a model of the error prone polymerase at issue, for example, using molecular graphics software such as the PyMOL viewer (open source, freely available on the World Wide Web at www(dot)pymol(dot)org) or Insight II (commercially available from Accelrys at (www (dot) accelrys (dot) com/products/insight). Alternatively, modeling can involve computer-assisted docking, molecular dynamics, free energy minimization, and/or similar calculations. Such modeling techniques have been well described in the literature; see, e.g., Babine and Abdel-Meguid (eds.) (2004) Protein Crystallography in Drug Design, Wiley-VCH, Weinheim; Lyne (2002) “Structure-based virtual screening: An overview” Drug Discov. Today 7:1047-1055; Molecular Modeling for Beginners, at (www (dot) usm (dot) maine (dot) edu/˜rhodes/SPVTut/index (dot) html; and Methods for Protein Simulations and Drug Design at (www (dot) dddc (dot) ac (dot) cn/embo04; and references therein. Software to facilitate such modeling is widely available, for example, the CHARMm simulation package, available academically from Harvard University or commercially from Accelrys (at www (dot) accelrys (dot) corn), the Discover simulation package (included in Insight II, supra), and Dynama (available at (www(dot) cs (dot) gsu (dot) edu/cscrwh/progs/progs (dot) html). See also an extensive list of modeling software at (www (dot) netsci (dot) org/Resources/Software/Modeling/MMMD/top (dot) html. Visual inspection and/or computational analysis of a polymerase model can identify relevant features of the active site region, including, for example, residues that can be bound by a putative modulator to sterically inhibit entry of a nucleotide analogue into the active site.

Genetic Polymerase Modulators

In addition to modulators that act on the Y polymerase proteins, such as the nucleobase and non-nucleobase inhibitors noted above, the activity of an error prone polymerase such as a Y family polymerase can be modulated by suppressing translation of Y polymerase mRNAs, e.g., using available RNAi or antisense strategies, and/or by modulating gene transcription, e.g., by inhibiting one or more transactivator of a Y polymerase gene. RNAi and antisense strategies have been used to successfully target, e.g., cancer cells (a preferred target of the invention); accordingly, it is possible to target RNAi or antisense therapeutics against Y DNA polymerases to target cancer cells of interest using essentially similar constructs, substituting shRNA or antisense genes targeted against Y polymerases.

For examples of formulations for the in vivo delivery of RNAi and antisense agents using, e.g., pegylated liposomes that comprise cell receptor targeting ligands to confer specificity of delivery see, e.g., Shi and Pardridge (2000) Non-invasive gene targeting to the brain. Proc Natl Acad Sci USA 97:7567-72; Zhang et al. (2002) “Receptor-mediated delivery of an antisense gene to human brain cancer cells” J Gene Med” 4:183-94; Zhang et al. (2004) “Intravenous RNA Interference Gene Therapy Targeting the Human Epidermal Growth Factor Receptor Prolongs Survival in Intracranial Brain Cancer” Clinical Cancer Research Vol. 10, 3667-3677; and Boado (2005) “RNA Interference and Nonviral Targeted Gene Therapy of Experimental Brain Cancer” NeuroRx. 2(1): 139-150. In general, these approaches can be used to target specific organs, cells, tumors, etc., by varying the cell receptor targeting ligand on the liposome. Delivery can be performed by intravenous delivery, even across the blood brain barrier (Zhang et al. 2004).

Further Details Regarding RNAi Design

The term “RNA interference” (“RNAi,” sometimes called RNA-mediated interference, post-transcriptional gene silencing, or quelling) refers to a phenomenon in which the presence of RNA, typically double-stranded RNA, in a cell results in inhibition of expression of a gene comprising a sequence identical, or nearly identical, to that of the double-stranded RNA. The double-stranded RNA responsible for inducing RNAi is called an “interfering RNA.” Expression of the gene is inhibited by the mechanism of RNAi as described below, in which the presence of the interfering RNA results in degradation of mRNA transcribed from the gene and thus in decreased levels of the mRNA and any encoded protein.

The mechanism of RNAi has been and is being extensively investigated in a number of organisms and cell types. See, for example, the following reviews: McManus and Sharp (2002) “Gene silencing in mammals by small interfering RNAs” Nature Reviews Genetics 3:737-747; Hutvagner and Zamore (2002) “RNAi: Nature abhors a double strand” Curr Opin Genet & Dev 200:225-232; Hannon (2002) “RNA interference” Nature 418:244-251; Agami (2002) “RNAi and related mechanisms and their potential use for therapy” Curr Opin Chem Biol 6:829-834; Tuschl and Borkhardt (2002) “Small interfering RNAs: A revolutionary tool for the analysis of gene function and gene therapy” Molecular Interventions 2:158-167; Nishikura (2001) “A short primer on RNAi: RNA-directed RNA polymerase acts as a key catalyst” Cell 107:415-418; and Zamore (2001) “RNA interference: Listening to the sound of silence” Nature Structural Biology 8:746-750. RNAi is also described in the patent literature; see, e.g., CA 2359180 by Kreutzer and Limmer entitled “Method and medicament for inhibiting the expression of a given gene”; WO 01/68836 by Beach et al. entitled “Methods and compositions for RNA interference”; WO 01/70949 by Graham et al. entitled “Genetic silencing”; and WO 01/75164 by Tuschl et al. entitled “RNA sequence-specific mediators of RNA interference.”

In brief, double-stranded RNA introduced into a cell (e.g., into the cytoplasm) is processed, for example by an RNAse DI-like enzyme called Dicer, into shorter double-stranded fragments called small interfering RNAs (siRNAs, also called short interfering RNAs). The length and nature of the siRNAs produced is dependent on the species of the cell, although typically siRNAs are 21-25 nucleotides long (e.g., an siRNA may have a 19 base pair duplex portion with two nucleotide 3′ overhangs at each end). Similar siRNAs can be produced in vitro (e.g., by chemical synthesis or in vitro transcription) and introduced into the cell to induce RNAi. The siRNA becomes associated with an RNA-induced silencing complex (RISC). Separation of the sense and antisense strands of the siRNA, and interaction of the siRNA antisense strand with its target mRNA through complementary base-pairing interactions, optionally occurs. Finally, the mRNA is cleaved and degraded.

Expression of a target Y DNA polymerase gene in a cell can thus be specifically inhibited by introducing an appropriately chosen double-stranded RNA into the cell (e.g., using available in vivo delivery systems, as noted above). Guidelines for the design of suitable interfering RNAs are known to those of skill in the art. For example, interfering RNAs are typically designed against exon sequences, rather than introns or untranslated regions. Characteristics of high efficiency interfering RNAs may vary by cell type. For example, although siRNAs may require 3′ overhangs and 5′ phosphates for most efficient induction of RNAi in Drosophila cells, in mammalian cells blunt ended siRNAs and/or RNAs lacking 5′ phosphates can induce RNAi as effectively as siRNAs with 3′ overhangs and/or 5′ phosphates (see, e.g., Czauderna et al. (2003) “Structural variations and stabilizing modifications of synthetic siRNAs in mammalian cells” Nucl Acids Res 31:2705-2716). As another example, since double-stranded RNAs greater than 30-80 base pairs long activate the antiviral interferon response in mammalian cells and result in non-specific silencing, interfering RNAs for use in mammalian cells are typically less than 30 base pairs (for example, Caplen et al. (2001) “Specific inhibition of gene expression by small double-stranded RNAs in invertebrate and vertebrate systems” Proc. Natl. Acad. Sci. USA 98:9742-9747, Elbashir et al. (2001) “Duplexes of 21-nucleotide RNAs mediate RNA interference in cultured mammalian cells” Nature 411:494-498 and Elbashir et al. (2002) “Analysis of gene function in somatic mammalian cells using small interfering RNAs” Methods 26:199-213 describe the use of 21 nucleotide siRNAs to specifically inhibit gene expression in mammalian cell lines, and Kim et al. (2005) “Synthetic dsRNA Dicer substrates enhance RNAi potency and efficacy” Nature Biotechnology 23:222-226 describes use of 25-30 nucleotide duplexes). The sense and antisense strands of a siRNA are typically, but not necessarily, completely complementary to each other over the double-stranded region of the siRNA (excluding any overhangs). The antisense strand is typically completely complementary to the target mRNA over the same region, although some nucleotide substitutions can be tolerated (e.g., a one or two nucleotide mismatch between the antisense strand and the mRNA can still result in RNAi, although at reduced efficiency). The ends of the double-stranded region are typically more tolerant to substitution than the middle; for example, as little as 15 by (base pairs) of complementarity between the antisense strand and the target mRNA in the context of a 21 mer with a 19 by double-stranded region has been shown to result in a functional siRNA (see, e.g., Czauderna et al. (2003) “Structural variations and stabilizing modifications of synthetic siRNAs in mammalian cells” Nucl Acids Res 31:2705-2716). Any overhangs can but need not be complementary to the target mRNA (e.g., a Y-polymerase gene); for example, TT (two 2′-deoxythymidines) overhangs are frequently used to reduce synthesis costs.

Although double-stranded RNAs (e.g., double-stranded siRNAs) were initially thought to be required to initiate RNAi, several recent reports indicate that the antisense strand of such siRNAs is sufficient to initiate RNAi. Single-stranded antisense siRNAs can initiate RNAi through the same pathway as double-stranded siRNAs (as evidenced, for example, by the appearance of specific mRNA endonucleolytic cleavage fragments). As for double-stranded interfering RNAs, characteristics of high-efficiency single-stranded siRNAs may vary by cell type (e.g., a 5′ phosphate may be required on the antisense strand for efficient induction of RNAi in some cell types, while a free 5′ hydroxyl is sufficient in other cell types capable of phosphorylating the hydroxyl). See, e.g., Martinez et al. (2002) “Single-stranded antisense siRNAs guide target RNA cleavage in RNAi” Cell 110:563-574; Amarzguioui et al. (2003) “Tolerance for mutations and chemical modifications in a siRNA” Nucl. Acids Res. 31:589-595; Holen et al. (2003) “Similar behavior of single-strand and double-strand siRNAs suggests that they act through a common RNAi pathway” Nucl. Acids Res. 31:2401-2407; and Schwarz et al. (2002) Mol. Cell. 10:537-548.

Due to currently unexplained differences in efficiency between siRNAs corresponding to different regions of a given target mRNA, several siRNAs are typically designed and tested against the target mRNA to determine which siRNA is most effective. Interfering RNAs can also be produced as small hairpin RNAs (shRNAs, also called short hairpin RNAs), which are processed in the cell into siRNA-like molecules that initiate RNAi (see, e.g., Siolas et al. (2005) “Synthetic shRNAs as potent RNAi triggers” Nature Biotechnology 23:227-231).

The presence of RNA, particularly double-stranded RNA, in a cell can result in inhibition of expression of a gene comprising a sequence identical or nearly identical to that of the RNA through mechanisms other than RNAi. For example, double-stranded RNAs that are partially complementary to a target mRNA can repress translation of the mRNA without affecting its stability. As another example, double-stranded RNAs can induce histone methylation and heterochromatin formation, leading to transcriptional silencing of a gene comprising a sequence identical or nearly identical to that of the RNA (see, e.g., Schramke and Allshire (2003) “Hairpin RNAs and retrotransposon LTRs effect RNAi and chromatin-based gene silencing” Science 301:1069-1074; Kawasaki and Taira (2004) “Induction of DNA methylation and gene silencing by short interfering RNAs in human cells” Nature 431:211-217; and Morris et al. (2004) “Small interfering RNA-induced transcriptional gene silencing in human cells” Science 305:1289-1292).

Short RNAs called microRNAs (miRNAs) have been identified in a variety of species. Typically, these endogenous RNAs are each transcribed as a long RNA and then processed to a pre-miRNA of approximately 60-75 nucleotides that forms an imperfect hairpin (stem-loop) structure. The pre-miRNA is typically then cleaved, e.g., by Dicer, to form the mature miRNA. Mature miRNAs are typically approximately 21-25 nucleotides in length, but can vary, e.g., from about 14 to about 25 or more nucleotides. Some, though not all, miRNAs have been shown to inhibit translation of mRNAs bearing partially complementary sequences. Such miRNAs contain one or more internal mismatches to the corresponding mRNA that are predicted to result in a bulge in the center of the duplex formed by the binding of the miRNA antisense strand to the mRNA. The miRNA typically forms approximately 14-17 Watson-Crick base pairs with the mRNA; additional wobble base pairs can also be formed. In addition, short synthetic double-stranded RNAs (e.g., similar to siRNAs) containing central mismatches to the corresponding mRNA have been shown to repress translation (but not initiate degradation) of the mRNA. See, for example, Zeng et al. (2003) “MicroRNAs and small interfering RNAs can inhibit mRNA expression by similar mechanisms” Proc. Natl. Acad. Sci. USA 100:9779-9784; Doench et al. (2003) “siRNAs can function as miRNAs” Genes & Dev. 17:438-442; Bartel and Bartel (2003) “MicroRNAs: At the root of plant development?” Plant Physiology 132:709-717; Schwarz and Zamore (2002) “Why do miRNAs live in the miRNP?” Genes & Dev. 16:1025-1031; Tang et al. (2003) “A biochemical framework for RNA silencing in plants” Genes & Dev. 17:49-63; Meister et al. (2004) “Sequence-specific inhibition of microRNA- and siRNA-induced RNA silencing” RNA 10:544-550; Nelson et al. (2003) “The microRNA world: Small is mighty” Trends Biochem. Sci. 28:534-540; Scacheri et al. (2004) “Short interfering RNAs can induce unexpected and divergent changes in the levels of untargeted proteins in mammalian cells” Proc. Natl. Acad. Sci. USA 101:1892-1897; Sempere et al. (2004) “Expression profiling of mammalian microRNAs uncovers a subset of brain-expressed microRNAs with possible roles in murine and human neuronal differentiation” Genome Biology 5:R13; Dykxhoom et al. (2003) “Killing the messenger: Short RNAs that silence gene expression” Nature Reviews Molec. and Cell Biol. 4:457-467; McManus (2003) “MicroRNAs and cancer” Semin Cancer Biol. 13:253-288; and Stark et al. (2003) “Identification of Drosophila microRNA targets” PLoS Biol. 1:E60.

The cellular machinery involved in translational repression of mRNAs by partially complementary RNAs (e.g., certain miRNAs) appears to partially overlap that involved in RNAi, although, as noted, translation of the mRNAs, not their stability, is affected and the mRNAs are typically not degraded.

The location and/or size of the bulge(s) formed when the antisense strand of the RNA binds the mRNA can affect the ability of the RNA to repress translation of the mRNA. Similarly, location and/or size of any bulges within the RNA itself can also affect efficiency of translational repression. See, e.g., the references above. Typically, translational repression is most effective when the antisense strand of the RNA is complementary to the 3′ untranslated region (3′ UTR) of the mRNA. Multiple repeats, e.g., tandem repeats, of the sequence complementary to the antisense strand of the RNA can also provide more effective translational repression; for example, some mRNAs that are translationally repressed by endogenous miRNAs contain 7-8 repeats of the miRNA binding sequence at their 3′ UTRs. It is worth noting that translational repression appears to be more dependent on concentration of the RNA than RNA interference does; translational repression is thought to involve binding of a single mRNA by each repressing RNA, while RNAi is thought to involve cleavage of multiple copies of the mRNA by a single siRNA-RISC complex.

Guidance for design of a suitable RNA to repress translation of a given target mRNA can be found in the literature (e.g., the references above and Doench and Sharp (2004) “Specificity of microRNA target selection in translational repression” Genes & Dev. 18:504-511; Rehmsmeier et al. (2004) “Fast and effective prediction of microRNA/target duplexes” RNA 10:1507-1517; Robins et al. (2005) “Incorporating structure to predict microRNA targets” Proc Natl Acad Sci 102:4006-4009; and Mattick and Makunin (2005) “Small regulatory RNAs in mammals” Hum. Mol. Genet. 14:R121-R132, among many others) and herein. However, due to differences in efficiency of translational repression between RNAs of different structure (e.g., bulge size, sequence, and/or location) and RNAs corresponding to different regions of the target mRNA, several RNAs are optionally designed and tested against the target mRNA to determine which is most effective at repressing translation of the target mRNA.

Further Details Regarding Antisense Modulators

The use of antisense nucleic acids for gene silencing is well known in the art and can be applied to the translational repression of Y DNA polymerases, according to the present invention. An antisense nucleic acid has a region of complementarity to a target nucleic acid, e.g., a target gene, mRNA, or cDNA. Typically, a nucleic acid comprising a nucleotide sequence in a complementary, antisense orientation with respect to a coding (sense) sequence of an endogenous gene is introduced into a cell. The antisense nucleic acid can be RNA, DNA, a PNA or any other appropriate molecule. A duplex can form between the antisense sequence and its complementary sense sequence, resulting in inactivation of the gene. The antisense nucleic acid can inhibit gene expression (transcription or translation) by forming a duplex with an RNA transcribed from the gene, by forming a triplex with duplex DNA, etc. An antisense nucleic acid can be produced, e.g., for any gene whose coding sequence is known or can be determined by a number of well-established techniques (e.g., chemical synthesis of an antisense RNA or oligonucleotide (optionally including modified nucleotides and/or linkages that increase resistance to degradation or improve cellular uptake) or in vitro transcription). Genes that encode antisense agents can also be specifically targeted in vivo, e.g., using the in vivo delivery methods noted above, e.g., to deliver the agents to cancer cells, bacterial cells, or the like. Antisense nucleic acids and their use are described, e.g., in U.S. Pat. No. 6,242,258 to Haselton and Alexander (Jun. 5, 2001) entitled “Methods for the selective regulation of DNA and RNA transcription and translation by photoactivation”; U.S. Pat. No. 6,500,615; U.S. Pat. No. 6,498,035; U.S. Pat. No. 6,395,544; U.S. Pat. No. 5,563,050; E. Schuch et al (1991) Symp Soc. Exp Biol 45:117-127; de Lange et al., (1995) Curr Top Microbiol Immunol 197:57-75; Hamilton et al. (1995) Curr Top Microbiol Immunol 197:77-89; Finnegan et al., (1996) Proc Natl Acad Sci USA 93:8449-8454; Uhlmann and A. Pepan (1990), Chem. Rev. 90:543; P. D. Cook (1991), Anti-Cancer Drug Design 6:585; J. Goodchild, Bioconjugate Chem. 1 (1990) 165; and, S. L. Beaucage and R. P. Iyer (1993), Tetrahedron 49:6123; and F. Eckstein, Ed. (1991), Oligonucleotides and Analogues—A Practical Approach, IRL Press.

Antibody Modulators

In another aspect, antibodies that are specific to error prone DNA polymerases (or complexes thereof) can be generated using methods that are well known. These can be administered by any available antibody administration method, and/or can be expressed in recombinant cassettes for expression in cells targeted by the cassette. The antibodies can also be utilized for detecting and/or purifying polypeptides or complexes of interest, e.g., in situ to monitor expression of DNA polymerase components.

As used herein, the term “antibody” includes, but is not limited to, polyclonal antibodies, monoclonal antibodies, humanized or chimeric antibodies and biologically functional antibody fragmentssufficient for binding of the antibody fragment to the polymerase protein or complex.

For the production of antibodies to a polypeptide encoded by a DNA polymerase, various host animals may be immunized by injection with the polypeptide, or a portion thereof. Such host animals may include, but are not limited to, rabbits, mice and rats. Various adjuvants may be used to enhance the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a polymerase, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature 256:495-497, 1975; and U.S. Pat. No. 4,376,110), the human B-cell hybridoma technique (Kosbor et al., Immunology Today 4:72, 1983; Cole et al., Proc. Nat'l. Acad. Sci. USA 80:2026-2030, 1983), and the EBV-hybridoma technique (Cole et al., Monoclonal Antibodies and Cancer Therapy, Alan R. Liss, Inc., pp. 77-96, 1985). For a modern review of the topic, see Howard and Kaser Making and Using Antibodies: A Practical Handbook ISBN: 0849335280 (2006). Such antibodies may be of any immunoglobulin class, including IgG, IgM, IgE, IgA, IgD, and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., Proc. Nat'l. Acad. Sci. USA 81:6851-6855, 1984; Neuberger et al., Nature 312:604-608, 1984; Takeda et al., Nature 314:452-454, 1985) by splicing the genes from a mouse antibody molecule of appropriate antigen specificity, together with genes from a human antibody molecule of appropriate biological activity, can be used to reduce unwanted immunogenicity (if the target patient is not human, an appropriate similar strategy is used, substituting antibody sequences for the target patient type). A chimeric antibody is a molecule in which different portions are derived from different animal species, such as those having a variable or hypervariable region derived from a murine mAb and a human immunoglobulin constant region.

Alternatively, techniques described for the production of single-chain antibodies (U.S. Pat. No. 4,946,778; Bird, Science 242:423-426, 1988; Huston et al., Proc. Nat'l. Acad. Sci. USA 85:5879-5883, 1988; and Ward et al., Nature 334:544-546, 1989) can be adapted to produce differentially expressed gene-single chain antibodies (this is useful, e.g., when delivery is by expression cassette, as described above). Single chain antibodies are formed by linking the heavy and light chain fragments of the Fv region via an amino acid bridge, resulting in a single-chain polypeptide.

Antibody fragments that recognize specific epitopes can be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science 246:1275-1281, 1989) to allow rapid and easy identification of monoclonal Fab fragments with the desired specificity. These fragments can also serve to inhibit DNA polymerase activity, e.g., by binding of the fragment to the polymerase.

Antibodies specific for DNA polymerase complexes are useful in modulating (e.g., blocking) DNA polymerase activity. In human therapeutic applications of such antibodies, e.g., where modulation of polymerase activity is desired, including any of those applications noted herein, antibodies can be humanized before use to further reduce immunogenicity (as compared to “chimeric” antibodies, described above). Thus, antibodies to polymerases can be generated by any available method, and subsequently humanized appropriately for use in vivo in humans. Many methods of humanizing antibodies are currently available, including those described in Howard and Kaser Making and Using Antibodies: A Practical Handbook ISBN: 0849335280 (2006). In typical approaches, humanized Abs are created by combining, at the genetic level, the complementarity-determining regions of a murine (or other mammalian) mAb with the framework sequences of a human Ab variable domain. This leads to a functional Ab with reduced immunogenic side effects in human therapy. Such techniques are generally described in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429. Methods of making “superhumanized” antibodies with still further reduced immunogenicity in humans are described in Tan et al. (2002) ““Superhumanized” Antibodies: Reduction of Immunogenic Potential by Complementarity-Determining Region Grafting with Human Germline Sequences: Application to an Anti-CD28,” The Journal of Immunology, 169:1119-1125. Any available humanization method can be applied to making humanized antibodies of the present invention, which can be used as polymerase modulators, as noted.

Expression Constructs for Expressed Modulators

In many embodiments, an expressed modulator of an error prone DNA polymerase is introduced into a cell in an expression construct. Examples include the genetic modulators noted (shRNAs and other RNAi agents, antisense, etc.), as well as other nucleic acid or polypeptide modulators. In some embodiments, expression constructs are transiently expressed in a cell, while in other embodiments, they are stably integrated into a cellular genome. Typically, transient expression is most appropriate where the target cell is an organ or cell of the patient that is not to be destroyed by the relevant therapeutic treatment, because permanently suppressing cellular function of the error prone DNA polymerases, e.g., via trans-lesion synthesis in a cell, may be deleterious to the cell. Where the target cell is to be destroyed by treatment (e.g., tumor cells, or pathogen cells), expression can be designed to be permanent.

Methods well known to those skilled in the art may be used to construct expression vectors containing sequences encoding, e.g., a genetic modulator and appropriate transcriptional and translational control elements for the expression construct. These methods include in vitro recombinant DNA techniques, synthetic techniques, and in vivo genetic recombination. Such techniques are described, for example, in Sambrook et al., Molecular Cloning—A Laboratory Manual (3^{rd} Ed.), Vol. 1-3, Cold Spring Harbor Laboratory, Cold Spring Harbor, 2000 and Ausubel, F. M. et al. (1989) Current Protocols in Molecular Biology, John Wiley & Sons, New York. N.Y., as currently supplemented.

Typical regulatory sequences of the expression vector can include enhancers, promoters, 5′ and 3′ untranslated regions, etc., e.g., which interact with host cellular proteins to carry out e.g., integration, transcription and/or translation. Depending on the vector system and cell utilized, any number of suitable transcription and translation elements, including constitutive and inducible promoters, can be used. One of skill is fully aware of expression vector constructs and construction methods; details can be found in Sambrook and Ausubel, as well as Berger and Kimmel, Guide to Molecular Cloning Techniques, Methods in Enzymology volume 152 Academic Press, Inc., San Diego, Calif. (Berger).

In addition, a plethora of kits are commercially available for the preparation, purification and cloning of plasmids or other relevant nucleic acids from cells, (see, e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms, or the like.

Typical vectors can contain transcription and translation terminators, transcription and translation initiation sequences, promoters useful for regulation of the expression of the particular target nucleic acid, integration sequences, etc. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both. See, Giliman & Smith, Gene 8:81 (1979); Roberts, et al., Nature, 328:731 (1987); Schneider, B., et al., Protein Expr. Purif 6435:10 (1995); Ausubel, Sambrook, Berger (above). A catalogue of Bacteria and Bacteriophages useful for cloning is provided, e.g., by the ATCC, e.g., The ATCC Catalogue of Bacteria and Bacteriophage published yearly by the ATCC. Additional basic procedures for sequencing, cloning and other aspects of molecular biology and underlying theoretical considerations are also found in Watson et al. (1992) Recombinant DNA Second Edition, Scientific American Books, NY. Suitable integration sequences (e.g., recombinase sites) can include FRT sites and loxP sites, which are recognized by the flp and cre recombinases, respectively (See U.S. Pat. No. 6,080,576, U.S. Pat. No. 5,434,066, and U.S. Pat. No. 4,959,317). The Cre-loxP and Flp-FRT recombinase systems are comprised of two basic elements: the recombinase enzyme and a small sequence of DNA that is specifically recognized by the particular recombinase. Both systems are capable of mediating the deletion, insertion, inversion, or translocation of associated DNA, depending on the orientation and location of the target sites. Recombinase systems are disclosed in U.S. Pat. No. 6,080,576, U.S. Pat. No. 5,434,066, and U.S. Pat. No. 4,959,317, and methods of using recombinase systems for gene disruption or replacement are provided in Joyner, A. L., Stricklett, P. K. and Torres, R. M. and Kuhn, R. In Laboratory Protocols for Conditional Gene Targeting (1997), Oxford University Press, New York.

In addition, essentially any nucleic acid (and virtually any labeled nucleic acid, whether standard or non-standard) can be custom or standard ordered from any of a variety of commercial sources, such as The Midland Certified Reagent Company (mcrc@oligos(dot)com), The Great American Gene Company (www(dot)genco(dot)com), ExpressGen Inc. (www(dot)expressgen(dot)com), Operon Technologies Inc. (Alameda, Calif.) and many others.

Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); and Atlas and Parks (eds) The Handbook of Microbiological Media (1993) CRC Press, Boca Raton, Fla.

Additional Details Regarding Pharmaceutical Modulator Compositions and Kits

The present invention includes formulations of DNA polymerases modulators, optionally in combination with one or more additional drug, as noted above. Formulations are typically adapted for particular uses and include, e.g., pharmaceutical compositions suitable for administration to a patient, i.e., physiologically compatible. Accordingly, compositions of the modulators/inhibitors will often further comprise one or more pharmaceutically acceptable buffers or carriers. In any of the compositions or formulations herein, the modulator/inhibitor can be formulated as a salt, a prodrug, or a metabolite. For further details regarding pharmaceutical compositions and formulations, see Remington, The Science and Practice of Pharmacy 21^{st} Edition (2005) Lippincott Williams & Wilkins ISBN-10: 0781746736; # ISBN-13: 978-0781746731.

Typically, compositions of the present invention can comprise a pharmaceutically effective buffer or carrier. As used herein, a “pharmaceutical acceptable carrier” is a pharmaceutically acceptable solvent, suspending agent or vehicle for delivering an inhibitor of the present invention to a microorganism, animal or human. The carrier may be, for example, gaseous, liquid or solid and is selected with the planned manner of administration and the particular modulator in mind. In general, where the compositions are nucleoside/nucleotides, appropriate formulations are already commercially available for either oral or intravenous delivery. Other small molecules, e.g., NNYPIs can be similarly delivered. Where genetic modulators are used, the modulators can be delivered by liposomal or viral vector formulations as noted above, typically in an appropriate buffer.

General examples of carriers include buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives.

Examples of pharmaceutically acceptable carriers for oral pharmaceutical formulations include lactose, sucrose, gelatin, agar and bulk powders. In certain embodiments, pharmaceutical compositions of the present invention are formulated as tablets or capsules for oral administration. Such tablets or capsules may be formulated for specific release characteristics, e.g., extended release capsules. In particular embodiments, wherein a composition of the invention comprises both an inhibitor of DNA repair or replication and another antimicrobial or cytotoxic agent, the composition may be formulated as a mixture or in layers, e.g., the antimicrobial or cytotoxic agent may be encapsulated by the inhibitor or vice versa.

Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also preferred carriers.

Examples of pharmaceutically acceptable carriers for topical formulations include: ointments, cream, suspensions, lotions, powder, solutions, pastes, gels, spray, aerosol or oil. Alternately, a formulation may comprise a transdermal patch or dressing such as a bandage impregnated with an active ingredient (e.g., inhibitor and/or second therapeutic agent such as an antibiotic) and optionally one or more carriers or diluents. Topical formulations can include a compound that enhances absorption or penetration of the active ingredient through the skin or other affected areas. Examples of such dermal penetration enhancers include dimethylsulfoxide and related analogues. In certain embodiments, to be administered in the form of a transdermal delivery system, the dosage administration will be continuous rather than intermittent throughout the dosage regimen.

Formulations suitable for parenteral administration include aqueous and non-aqueous formulations isotonic with the blood of the intended recipient; and aqueous and non-aqueous sterile suspensions which may include suspending systems designed to target the compound to blood components of one or more organs. The formulations may be presented in unit-dose or multi-dose sealed containers, for example, ampoules or vials. Extemporaneous injection solutions and suspensions may be prepared from sterile powders, granules and tablets of the kind previously described. Parenteral and intravenous formulation may include minerals and other materials to make them compatible with the type of injection or delivery system chosen.

Commonly used pharmaceutically acceptable carriers for parenteral administration includes, water, a suitable oil, saline, aqueous dextrose (glucose), or related sugar solutions and glycols such as propylene glycol or polyethylene glycols. Solutions for parenteral administration preferably contain a water soluble salt of the active ingredient, suitable stabilizing agents and, if necessary, buffer substances, antioxidizing agents, such as sodium bisulfite, sodium sulfite, or ascorbic acid, either alone or combined, are suitable stabilizing agents. Citric acid salts and sodium EDTA may also be used as carriers. In addition, parenteral solutions may contain preservatives, such as benzalkonium chloride, methyl- or propyl-paraben, or chlorobutanol. Suitable pharmaceutical carriers are described in Remington, cited above.

In addition to human administration, the present invention additionally contemplates inhibitors formulated for veterinary administration, i.e., by methods conventional in the art.

The compositions and pharmaceutical formulation herein can be administered to an organism by any means known in the art. Routes for administering the compositions and pharmaceutical formulations herein to an animal, such as a human, include parenterally, intravenously, intramuscularly, orally, by inhalation, topically, vaginally, rectally, nasally, buccally, transdermally, as eye drops, or by an implanted reservoir external pump or catheter.

Injectable formulations can be prepared in conventional forms, either as liquid solutions or suspensions; as solid forms suitable for solubilization or suspension in liquid prior to injection; or as emulsions. Preferably, sterile injectable suspensions are formulated according to techniques known in the art using suitable pharmaceutically acceptable carriers and other optional components, as described above.

Parenteral administration may be carried out in any number of ways, but it is preferred that a syringe, catheter, or similar device, be used to effect parenteral administration of the formulations described herein. The formulation may be injected systemically such that the active agent travels substantially throughout the entire bloodstream.

Also, the formulation may also be injected locally to a target site, e.g., injected to a specific portion of the body comprising the target cell population. An advantage of local administration via injection is that it limits or avoids exposure of the entire body to the active agent(s) (e.g., inhibitors and/or other therapeutic agents). It must be noted that in the present context, the term local administration includes regional administration, e.g., administration of a formulation directed to a portion of the body through delivery to a blood vessel serving that body zone. Local delivery may be direct, e.g., intratumoral. Local delivery may also be nearly direct, i.e., intralesional or intraperitoneal, that is, to an area that is sufficiently close to a tumor or site of infection so that the inhibitor exhibits the desired pharmacological activity. Thus, when local delivery is desired, the pharmaceutical formulations are preferably delivered intralesionally, intratumorally, or intraperitoneally.

In certain embodiments, the pharmaceutical compositions are in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet, or it can be the appropriate number of any of these packaged forms.

Pharmaceutical compositions of the present invention will typically comprise an amount of inhibitor that is sufficient to achieve a reduction in the emergence of drug resistance in the patient. The actual effective amount will depend upon the condition being treated, the route of administration, the drug treatment used to treat the condition, and the medical history of the patient. Determination of the effective amount is well within the capabilities of those skilled in the art. The effective amount for use in humans can be determined from animal models. For example, a dose for humans can be formulated to achieve circulating concentrations that have been found to be effective in animals. The effective amount of an inhibitor can vary if the inhibitor is coformulated with another therapeutic agent (e.g., antimicrobial or cytotoxic agent or compound, such as an antibiotic, an antineoplastic agent, an antiviral agent, an antiprotozoan agent, etc.).

In particular embodiments, an effective amount of an active ingredient (e.g., a modulator or second therapeutic agent/drug such as an antibiotic or chemotherapeutic) is from about 0.0001 mg to about 500 mg active agent per kilogram body weight of a patient, more preferably from about 0.001 to about 250 mg active agent per kilogram body weight of the patient, still more preferably from about 0.01 mg to about 100 mg active agent per kilogram body weight of the patient, yet still more preferably from about 0.5 mg to about 50 mg active agent per kilogram body weight of the patient, and most preferably from about 1 mg to about 15 mg active agent per kilogram body weight of the patient. In terms of weight percentage, a pharmaceutical formulation of an active agent (e.g., an inhibitor or second therapeutic agent) preferably comprises of an amount from about 0.0001 wt. % to about 10 wt. %, more preferably from about 0.001 wt. % to about 1 wt. %, and more preferably from about 0.01 wt. % to about 0.5 wt. %.

### Screening Assays to Test Potential Modulators

Potential modulators of the invention can be identified in libraries of compounds using any of a variety of approaches, depending on the type of modulator at issue and the available assay instrumentation of the practitioner.

For chain terminating nucleobases, the ability of the error prone DNA polymerase to incorporate the chain terminating nucleobase is measured. Both the absolute ability of the enzyme to incorporate the terminating nucleobase and the relative rate at which the error prone polymerase incorporates the nucleobase can be measured. Assays to measure incorporation can include in vitro assays that monitors the presence of chain terminated nucleotide products, or, e.g., by measuring changes in the rate at which resistance forms against an antibiotic of interest, e.g., in a bacterial cell-based survival assay (examples of such assays are described in the Examples section below). In the later case, mutagens such as ultraviolet light can be used to induce the formation of antibiotic resistant bacterial colonies; the rate at which such colonies form in the presence and absence of a putative modulator of interest is measured. Changes in the rate of resistance formation indicate that the putative modulator may act on a Y-family DNA polymerase.

In vitro assays can also be used to measure polymerase activity against such nucleobase analogs. For example, in one simple assay, the nucleobase analogue is labeled and incorporation of the analog is measured by detecting incorporating of the analogue into primer extension products. Similarly, premature termination of primer extension products can be detected in the presence of the nucleobase analogues, by standard nucleic acid labeling and size detection methods (electrophoresis, etc.).

In general, high-throughput screening of libraries of compounds for modulatory activity against a target error prone (e.g., Y family) DNA polymerase can be measured by any available method. These libraries can include random sets of compounds, structurally related compounds, or designed/modeled compounds made based upon structural consideration of the polymerase. These assays can measure (a.) the rate at which nucleobase analogue modulators are incorporated into primer extension products, or changes in the rate at which the target polymerase enzyme incorporates natural nucleotides. These measurements can be direct (e.g., by monitoring primer extension products), or the assays can indirectly measure this activity, e.g., by monitoring changes in mutation rates.

As noted herein, specific Y family DNA polymerase modulators are described, such as AZT and other chain terminating nucleotide/nucleosides. These available modulators can be used as positive controls in any assay that measures polymerase activity, and/or the relative activity of any putative modulator can be compared to these example modulators.

Further details regarding screening methodology applicable to the invention are presented below and are also found in Cirz et al. (2005) “Inhibition of Mutation and Combating the Evolution of Antibiotic Resistance,” PLoS Biology 3(6):1024-1033; Romesberg et al. COMPOSITIONS AND METHODS TO REDUCE MUTAGENESIS WO2005/056754; and Romesberg et al. COMPOSITIONS AND METHODS FOR ENHANCING DRUG SENSITIVITY AND TREATING DRUG RESISTANT INFECTIONS AND DISEASES WO2006/108075.

Determining Kinetic Parameters

Changes in Y DNA polymerase activity can include changes in standard kinetic parameters, and/or changes in mutation rate, survival rate, or the like. The polymerases of the invention can be screened or otherwise tested to determine whether the polymerase displays a modified enzymatic activity in the presence of a putative modulator as compared to the polymerase in the absence of the modulator. These tests can be conducted by survival assays as noted above and as exemplified in the Examples sections herein, or they can be conducted in vitro, or using simple cell-based assays to measure kinetic parameters. It is useful to measure survival and/or kinetic parameters to determine the quantitative effects on polymerase function caused by a modulator of interest. For example, k_{cat}, Kₘ, Vₘₐₓ, k_{cat}/Kₘ, Vₘₐₓ/Kₘ, kₚₒₗ, and/or K_{d} of the DNA polymerase for a nucleotide in the presence of the modulator can be determined, using available assays that measure nucleotide uptake. Modulators that make the polymerase less able to incorporate natural nucleotides, or more able to incorporate unnatural nucleotide analogs are useful. Similarly, nucleotide analogs are, themselves, modulators of polymerase activity; analogs that are incorporated as approximately as well as natural nucleotides are more useful than analogs that the polymerase discriminates against.

As is well-known in the art, for enzymes obeying simple Michaelis-Menten kinetics, kinetic parameters are readily derived from rates of catalysis measured at different substrate concentrations. The Michaelis-Menten equation, V=Vₘₐₓ[S]([S]+Kₘ)⁻¹, relates the concentration of uncombined substrate ([S], approximated by the total substrate concentration), the maximal rate (Vₘₐₓ, attained when the enzyme is saturated with substrate), and the Michaelis constant (Kₘ, equal to the substrate concentration at which the reaction rate is half of its maximal value), to the reaction rate (V).

For many enzymes, Kₘ is equal to the dissociation constant of the enzyme-substrate complex and is thus a measure of the strength of the enzyme-substrate complex. For such an enzyme, in a comparison of Kₘs, a lower Kₘ represents a complex with stronger binding, while a higher Km represents a complex with weaker binding. The ratio k_{cat}/Kₘ, sometimes called the specificity constant, represents the apparent rate constant for combination of substrate with free enzyme. The larger the specificity constant, the more efficient the enzyme is in binding the substrate and converting it to product.

k_{cat} (also called the turnover number of the enzyme) can be determined if the total enzyme concentration ([E_{T}], i.e., the concentration of active sites) is known, since Vₘₐₓ=k_{cat}[E_{T}]. For situations in which the total enzyme concentration is difficult to measure, the ratio Vₘₐₓ/Kₘ is often used instead as a measure of efficiency. Kₘ and Vₘₐₓ can be determined, for example, from a Lineweaver-Burk plot of 1/V against 1/[S], where the y intercept represents 1/Vₘₐₓ, the x intercept −1/Kₘ, and the slope Kₘ/Vₘₐₓ, or from an Eadie-Hofstee plot of V against V/[S], where the y intercept represents Vₘₐₓ, the x intercept Vₘₐₓ/Kₘ, and the slope −Kₘ. Software packages such as KinetAsyst™ or Enzfit (Biosoft, Cambridge, UK) can facilitate the determination of kinetic parameters from catalytic rate data.

For a more thorough discussion of enzyme kinetics, see, e.g., Berg, Tymoczko, and Stryer (2002) Biochemistry, Fifth Edition, W. H. Freeman; Creighton (1984) Proteins: Structures and Molecular Principles, W. H. Freeman; and Fersht (1985) Enzyme Structure and Mechanism, Second Edition, W. H. Freeman.

Library Screening Strategies

In one embodiment, modulators such as inhibitors are identified by screening libraries of molecules or chemical compounds, e.g., small molecules for action against the relevant polymerase target. Such libraries and methods of screening the same are known in the art and include: biological libraries, natural products libraries, spatially addressable parallel solid phase or solution phase libraries, synthetic library methods requiring deconvolution, the ‘one-bead one-compound’ library method, and synthetic library methods using affinity chromatography selection. The biological library approach includes polypeptide libraries, while the other four approaches are applicable to polypeptide, non-peptide oligomer or small molecule libraries of compounds. See Lam, K. S. (1997) Anticancer Drug Des. 12:145. In certain embodiments, screening of libraries is performed using an array or microarray, which permits the testing of multiple compounds, e.g., small molecules, polypeptides, or antibodies) simultaneously. In particular embodiments, library screening includes high throughput screening.

In one embodiment, inhibitors are identified using an Automated Ligand Identification System (referred to herein as “ALIS”). See, e.g., U.S. Pat. Nos. 6,721,665, 6,714,875, 6,694,267, 6,691,046, 6,581,013, 6,207,861, and 6,147,344. ALIS is a high-throughput technique for the identification of small molecules that bind to proteins of interest (e.g., Y family DNA polymerases). Small molecules found to bind tightly to a polymerase target can then be tested for their ability to inhibit the biochemical activity of that protein; specificity can be determined by comparing the activity of the molecule against the error prone polymerase as compared to other classes of polymerases.

Thus, in some embodiments, a target protein (e.g., Y family DNA polymerase) is mixed with pools of small molecules. Typically, more than 1,000 pools are used, more preferably more than 2,000 pools are used, more preferably more than 3,000 pools are used, or more preferably, more than 10,000 pools are used. Each pool contains approximately, 1,000 compounds, more preferably approximately 2,500 compounds, or more preferably approximately 5,000 compounds that are ‘mass encoded,’ meaning that their precise molecular structure can be determined using only their mass and knowledge of the chemical library.

The small molecules and proteins are mixed together and allowed to come to equilibrium. The mixture is rapidly cooled to trap bound complexes and subject to rapid size exclusion chromatography (SEC). Small molecules that bind tightly to the protein of interest will be co-excluded with the protein during SEC. Mass spectroscopic analysis is performed to determine the masses of all small molecules found to bind the protein. Measurement of these masses allows for the rapid determination of the molecular structures of the small molecules.

In certain embodiments, such screening methods further comprise testing agents identified based upon their ability to bind an error prone DNA polymerase for their ability to reduce genomic mutation rates, e.g., using reversion mutation assays, or assays that test for the emergence of antibiotic resistance (see also, the Examples herein).

Libraries of Modulators

Libraries of putative or actual modulators can be either physical or logical in nature. Moreover, any of a wide variety of library formats can be used. For example, compounds can be fixed to solid surfaces in arrays, or liquid phase arrays of modulator compounds (e.g., in microwell plates) can be constructed for convenient high-throughput fluid manipulations of solutions comprising the compounds. Liquid, emulsion, or gel-phase libraries of cells that produce compounds can also be constructed, e.g., in microwell plates, or on agar plates. Expression or phage display libraries of polymerase domains (e.g., including the active site region) can be also be produced to facilitate binding studies between the polymerase and modulator compound. Instructions in making and using libraries can be found, e.g., in Sambrook, Ausubel and Berger, referenced herein.

Essentially any available compound library can be screened in such a high-throughput format against a biological or biochemical sample, such as a cell expressing a Y family DNA polymerase, and activity of the library members against the polymerase can be assessed, optionally in a high-throughput fashion.

For example, with regard to the compositions, systems and methods of the invention, the particular libraries of potential modulator compounds can be any compound library that now exists, e.g., those that are commercially available, or that are proprietary. A number of libraries of test compounds exist, e.g., those from Sigma (St. Louis Mo.), and Aldrich (St. Louis Mo.). Other current compound library providers include Actimol (Newark Del.), providing e.g., the Actiprobe 10 and Actiprobe 25 libraries of 10,000 and 25,000 compounds, respectively; BioMol (Philadelphia, Pa.), providing a variety of libraries, including natural compound libraries; Enamine (Kiev, Ukranie) which produces custom libraries of billions of compounds from thousands of different building blocks, TimTec (Newark Del.), which produces general screening stock compound libraries containing >100,000 compounds, as well as template-based libraries with common heterocyclic lattices, libraries for targeted mechanism based selections, including polymerase modulators, privileged structure libraries that include compounds containing chemical motifs that are more frequently associated with higher biological activity than other structures, diversity libraries that include compounds pre-selected from available stocks of compounds with maximum chemical diversity, plant extract libraries, natural products and natural product-derived libraries, etc; AnalytiCon Discovery (Germany) including NatDiverse (natural product analogue screening compounds) and MEGAbolite (natural product screening compounds); Chembridge (San Diego, Calif.) including a wide array of targeted or general and custom or stock libraries; ChemDiv (San Diego, Calif.) providing a variety of compound diversity libraries including CombiLab and the International Diversity Collection; Comgenix (Hungary) including ActiVerse™ libraries; MicroSource (Gaylordsville, Conn.) including natural libraries, agro libraries, the NINDS custom library, the genesis plus library and others; Polyphor (Switzerland) including privileged core structures as well as novel scaffolds; Prestwick Chemical (Washington D.C.), including the Prestwick chemical collection and others that are pre-screened for biotolerance; Tripos (St. Louis, Mo.), including large lead screening libraries; and many others. Academic institutions such as the Zelinsky Institute of Organic Chemistry (Russian Federation) also provide libraries of considerable structural diversity that can be screened in the methods of the invention.

For the generation of libraries involving fluid transfer to or from microtiter plates, a fluid handling station is optionally used. Several “off the shelf” fluid handling stations for performing such transfers are commercially available, including e.g., the Zymate systems from Caliper Life Sciences (Hopkinton, Mass.) and other stations which utilize automatic pipettors, e.g., in conjunction with the robotics for plate movement (e.g., the ORCA® robot, which is used in a variety of laboratory systems available, e.g., from Beckman Coulter, Inc. (Fullerton, Calif.). In an alternate embodiment, fluid handling is performed in microchips, e.g., involving transfer of materials from microwell plates or other wells through microchannels on the chips to destination sites (microchannel regions, wells, chambers or the like). Commercially available microfluidic systems include those from Hewlett-Packard/Agilent Technologies (e.g., the HP2100 bioanalyzer) and the Caliper High Throughput Screening System. The Caliper High Throughput Screening System provides one example interface between standard microwell library formats and Labchip technologies. Furthermore, the patent and technical literature includes many examples of microfluidic systems which can interface directly with microwell plates for fluid handling.

Further details regarding assay formats, as well as general molecular biological methods generally useful in the present invention include Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2006) (“Ausubel”)) and PCR Protocols A Guide to Methods and Applications (Innis et al. eds) Academic Press Inc. San Diego, Calif. (1990) (“Innis”).

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

### Example 1

### AZT Inhibits Induced Mutagenesis in E. Coli

Genomic mutation underlies numerous processes related to human disease, including, for example, antibiotic and chemotherapeutic resistance, oncogenesis, tumor progression, and ageing. DNA is constantly under metabolic and environmental assault (Lindahl, T., Nature 362, 709 (1993)), which adds significantly to the background mutagenesis rate and, thereby, likely contributes importantly to the above disease processes. In both prokaryotes and eukaryotes, mounting evidence suggests that DNA damage does not directly generate mutations; rather, DNA damage induces the production and/or activation of specific enzymes, the error-prone DNA polymerases, which actually install mutations (Friedberg, E. C., et al., Science 296, 1627 (2002)). Most likely having evolved to enable synthesis across otherwise fatal damage-induced DNA lesions, via a repair mechanism known as translesion synthesis (TLS), error-prone polymerases leave mutations in their wake when replicating either undamaged or non-cognate lesion templates.

In unicellular organisms, error-prone polymerase-mediated mutagenesis has, perhaps, come to serve as a mechanism for creating genetic diversity in stressful times and, thereby, to drive evolution (Lombardo, M.-J. and Rosenberg, S. M., J. Genet. 78, 13-21 (1999); Friedberg, E. C., et al., Science 296, 1627 (2002)). Indeed, error-prone polymerases have recently been implicated in the evolution of resistance to ciprofloxacin and rifampicin in E. coli (Cirz, R. T., Chin, J. K., Andes, D. R., de Crecy-Legard, V., Craig, W. A., Romesberg, F. E. PLoS Biol, 2005, 3:e176) and to the persistence, virulence, and rifampicin-resistance in M. tuberculosis. In humans, however, somatic mutations provide no evolutionary benefit and pose tremendous cancer risk. Consequently, human cells have multiple, highly-specialized error-prone polymerases that maintain damage-induced mutagenesis at low levels. But recent evidence suggests human error-prone polymerases are nonetheless involved in somatic hypermutation, most notably in the evolution of immunoglobulin genes (Boshoff, H. I., Reed, M. B., Barry, C. E. 3^{rd}, and Mizrahi, V., Cell, 2003, 18: 183-193). Selective inhibition of these polymerases should prove useful in situations where mutation is harmful or unwanted.

In response to DNA damage, E. coli up-regulates more than 40 genes collectively referred to as the mutagenic SOS response. Two of these genes, umuC and umuD, encode DNA Pol V, a member of the Y-family of error-prone DNA polymerases (Tang M, Shen X, Frank E, O'Donnell M, Woodgate R, Goodman F, (1999) Proc Natl Acad Sci USA 96, 8919-8924). Pol V exhibits behaviors common to all error-prone DNA polymerases, notably a much-reduced fidelity on undamaged DNA templates, a lack of 3′→5′ exonuclease proofreading activity, and an active role in TLS, specifically over UV-induced DNA lesions and abasic sites. The importance of this error prone polymerase to induced mutation in E. coli is confirmed by experiments that show that deletion of umuC and umuD renders E. coli nonmutable to UV light and to many chemical mutagens.

The above indicates that induced mutagenesis in E. coli could be blocked by either inhibiting the action of Pol V or its up-regulation after DNA damage. Pol V is similar to viral DNA polymerases, for example reverse transcriptase from HIV-1, in that both have low fidelity and lack exonuclease proofreading ability (Goodman, M. F., Annu. Rev. Biochem., 2002, 71:17-50; Roberts, J D, Bebenek, K, Kunkel, T A, Science, 1988, 242: 1171-3). The chain-terminating nucleoside analogs, such as AZT (zidovudine, 3′-azido-2′-deoxythymidine), exploit these characteristics for their antiretroviral activity; once activated to the corresponding triphosphates, they become readily incorporated into DNA due to the low fidelity and then terminate synthesis, as the viral polymerase cannot excise them (Ruprecht, R M, O'Brien, L G, Rossoni, L D, Nusinoff-Lehrman, S, Nature, 1986, 323: 467-9). Several members of this drug class, including AZT and d4T (stavudine, 2′,3′-didehydro-3′-deoxythymidine), are metabolized to the nucleotide triphosphate by, and have antibacterial activity against, most gram-negative bacteria (Elwell et al., Antimicrob. Agents Chemother. 1987, 31: 274-280).

The experiments described below demonstrate that these analogs selectively inhibit the bacterial error-prone polymerases and eliminate damage-induced mutation at a concentration below their antibacterial activity, and in particular, this is indeed the case in E. coli. These results establish AZT as the first known inhibitor of induced genomic mutation, and indicate that it and other inhibitors of the error-prone polymerases may have important applications in inhibiting induced mutation in other organisms.

Materials. Solid media used was Lennox LB plus 1.6% agar (Miller, J H. A Short Course in Bacterial Genetics, 1992, 150-156); liquid media was Miller L B (Miller, J H. A Short Course in Bacterial Genetics, 1992, 150-156). All bacteria were grown at 37° C. Ciprofloxacin was purchased from MP Biomedials; Triclosan (Irgasan) from Fluka; AZT from Sigma; MMS from Acros Organics.

Strains. Wild-type bacteria was MG1655 K12 E. coli. The previously constructed E. coli RTC0005 ΔumuDC was used. Strain CC102 use from the Miller lab.

UV Mutagenesis. A reported UV mutagenesis procedure was followed (Miller, J H. A Short Course in Bacterial Genetics, 1992, 150-156). Briefly, 3 18 hour overnight cultures were diluted 1:100 into 5 mL fresh LB and grown to OD₆₀₀=0.4. The cultures were pelleted at 4° C., the supernatant decanted, and then re-suspended in 5 mL cold 9 mg/mL NaCl. 4.5 mL was transferred to a Petri dish. The remaining 0.5 mL was serially diluted and plated on LB (and LB+2.5 ng/mL AZT) for viable cell counts without UV treatment. Each Petri dish was irradiated for the indicated amount of time at 0.86 J/m²s as measured by UVX Digital Radiometer (UVP, Inc., Upland, Calif.). Aliquots were serially diluted and plated on LB (and LB+2.5 ng/mL AZT) for viable cell counts after UV treatment. 3.0 mL was transferred to 57 mL of LB in a 250 mL Erlenmeyer Flask with the indicated concentration of AZT, covered with aluminum foil, and shaken for 18 hours. 100 μl of the saturated cultures was plated two times on ciprofloxacin 100 ng/mL and triclosan 1 μg/mL. For strain CC102, the cultures were also plated on lactose minimal media prepared as described (Miller, J H. A Short Course in Bacterial Genetics, 1992, 150-156). The saturated cultures were also serially diluted and plated on LB for viable cell counts. The colonies appearing on the selection plates were counted after 48 hours. The counts from two plates for each culture were averaged, and the median number of mutants in 10⁸ viable cells of the three cultures was determined. This experiment was repeated 3 or more times for each concentration of AZT and UV light dosage, and the median of number of mutants in 10⁸ viable cells and standard deviation was determined.

MMS Mutagenesis. 5 18 hour overnight cultures were diluted 1:100 into 5 mL fresh LB and grown to OD₆₀₀=0.40. The cultures were centrifuged at 4° C. for 10 minutes, the supernatant was decanted, and the pellets were re-suspended in 5 mL cold PBS. 8.5 ul of MMS (or no MMS) was added to each culture, and each culture was shaken at 37° C. at 125 rpm for 15 minutes. The cells were then spun and washed with 5 mL cold PBS twice, and then re-suspended in cold PBS. Aliquots from these cultures were diluted appropriated and plated on LB and LB+2.5 ng/mL AZT for viable cell counts. 1.25 mL of each culture was added to 23.75 mL LB in a 50 mL Falcon tube with the indicated concentration of AZT and grown overnight for 18 h. 100 μl of each saturated culture was plated two times on ciprofloxacin 100 ng/mL. Aliquots were diluted appropriately and plated for viable cell counts. The colonies appearing on the selection plates were counted after 48 hours. The counts for the two plates from each culture were averaged, and the median number of mutants in 10⁸ viable cells was determined. This experiment was repeated 3 or more times for each concentration of AZT and MMS, and the median of number of mutants in 10⁸ viable cells and standard deviation was determined.

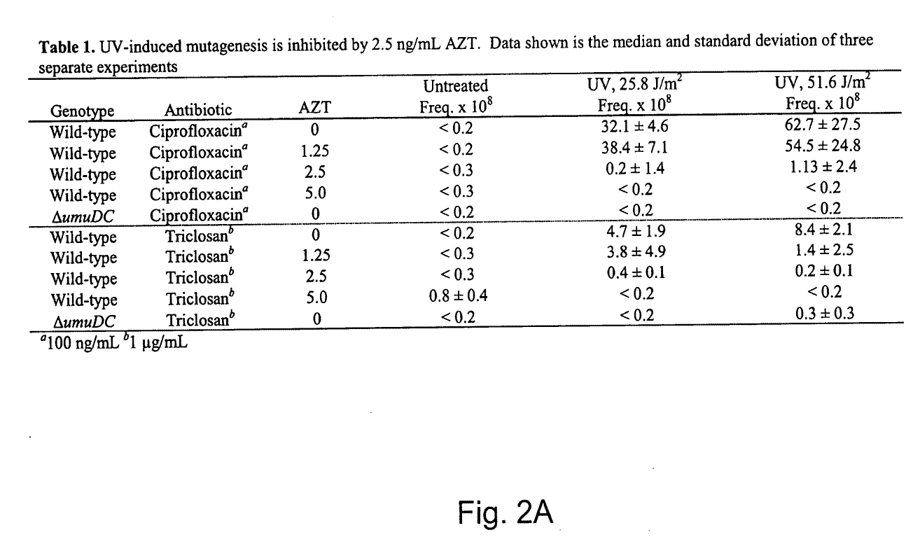

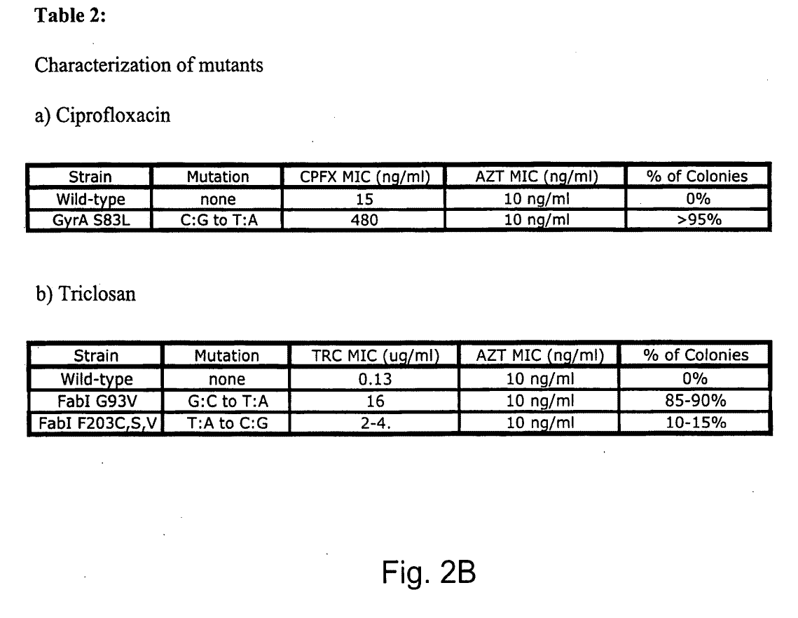

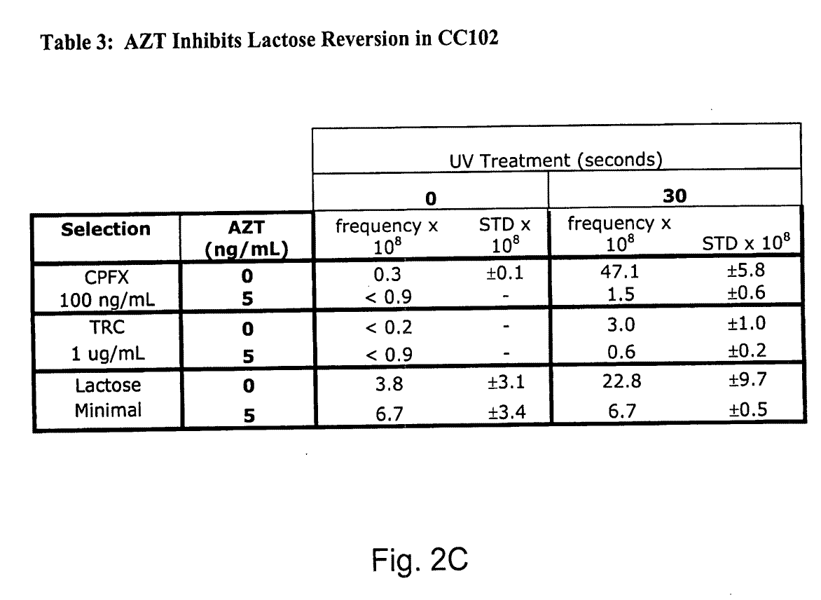

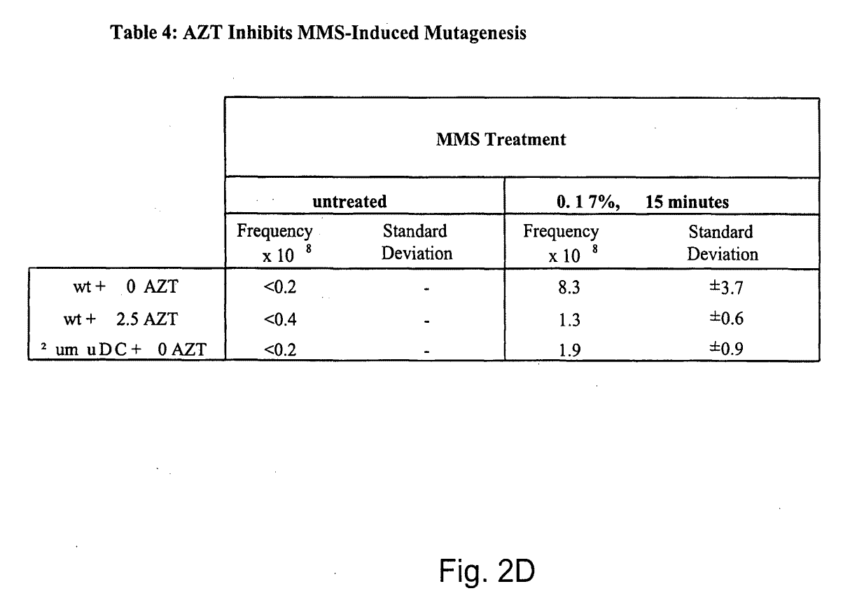

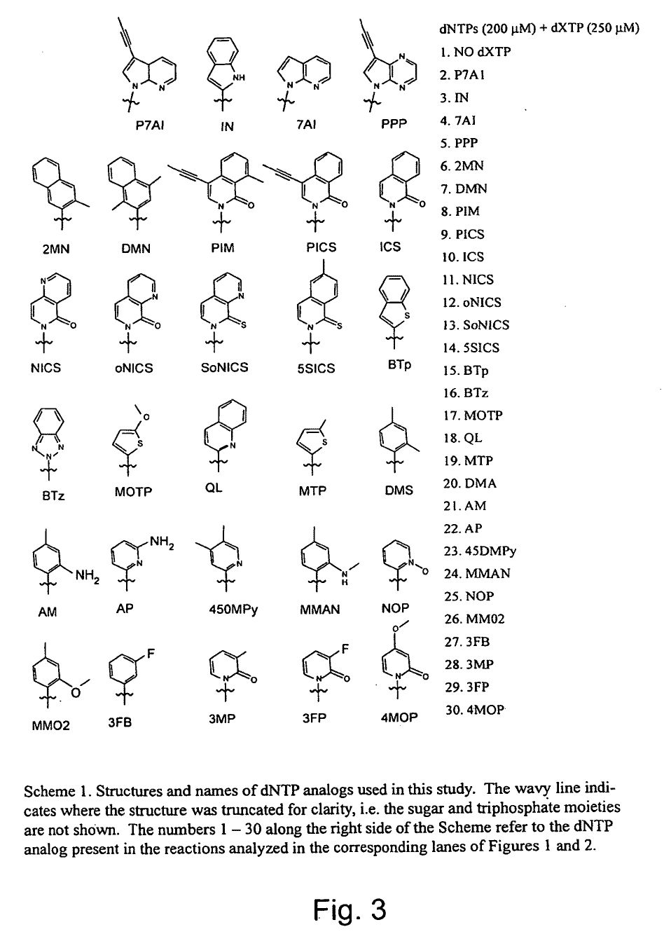

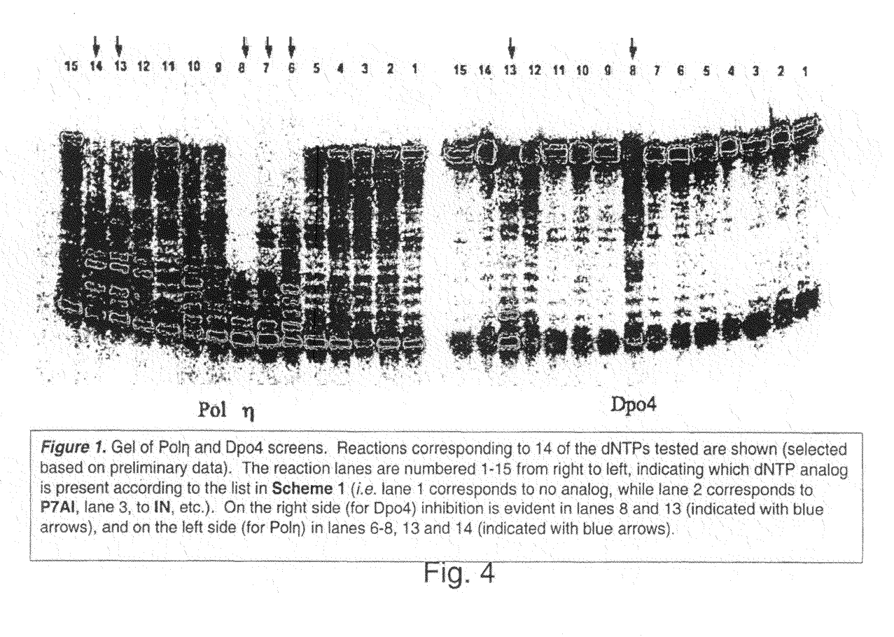

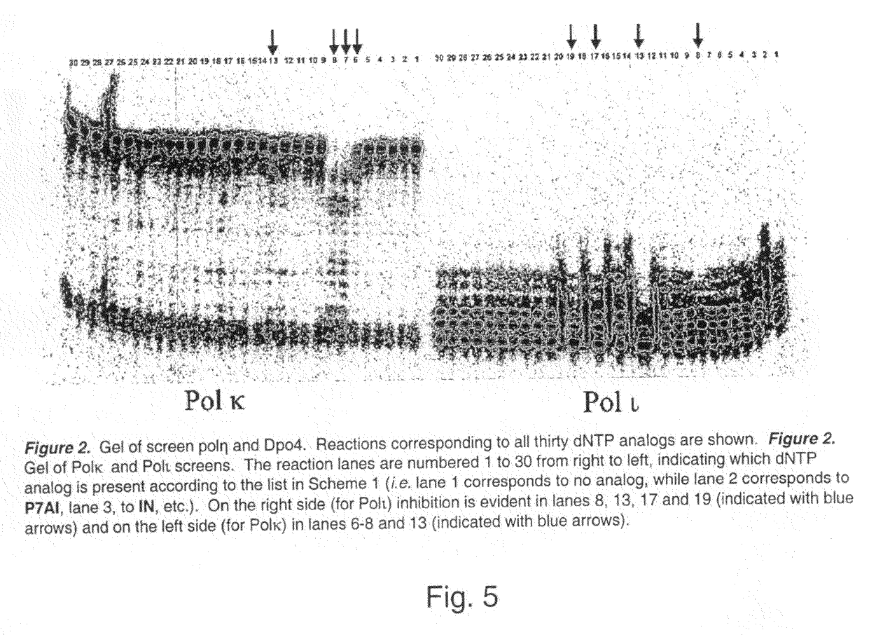

AZT inhibits UV-induced antibiotic resistance. Using an established UV mutagenesis procedure for E. coli (Miller, J. H., J Mol Biol 182: 45-68 (1985)), it was confirmed that UV irradiation robustly induced mutations that confered resistance to ciprofloxacin or triclosan (FIG. 2A). The procedure involved recovering irradiated cultures in rich media before selecting for antibiotic resistant mutants. To explore whether AZT could inhibit UV-induced mutation, cultures in rich media containing sub-lethal concentrations of AZT (0.625, 1.25, 2.5, 5 ng/mL) were recovered. Remarkably, UV-induced mutagenesis was essentially eliminated in cultures treated with 2.5 ng/mL AZT, as the frequency of both ciprofloxacin- and triclosan-resistant mutants was reduced to spontaneous background levels (FIG. 2A). 2.5 ng/mL AZT, ¼ of the MIC, was the minimal concentration required to eliminate induced mutation after treatment with UV energy fluences of either 25.8 or 51.6 J/m² (FIG. 2A). Genetic deletion of Pol V also eliminated UV-induced mutagenesis, as again only spontaneous frequencies of ciprofloxacin- and triclosan-resistant mutants were observed after treatment of a ΔumuDC E. coli strain with UV light (FIG. 2A).

The above results demonstrate that AZT has a potent anti-mutagenesis effect and indicate that it most likely mediates this effect though inhibition of Pol V. It was confirmed that other chain-terminating nucleoside analogs metabolized by E. coli should have similar effects by demonstrating that d4T (6 μg/mL) also inhibits the UV-induced generation of mutations that confer resistance to ciprofloxacin.

Characterization of mutants. The MICs were determined of over one hundred isolated antibiotic-resistant colonies. All of the ciprofloxacin-resistant mutants had MICs of 480 ng/mL (FIG. 2B). The ciprofloxacin resistance mutants contained a CG to TA transition translating into a Ser 83→Leu mutation in DNA gyrase. This mutation is known to provide the greatest ciprofloxacin resistance obtainable in a single step (Drlica K, Zhao X (1997) Microbiol Mol Biol Rev 61: 377-392). Interestingly, CG to TA transitions are the mutations most frequently induced by UV light, but sequence context can lead to variations from this rule (Miller, J. H. (1985) J Mol Biol 182: 45-68).

The vast majority of isolated triclosan-resistant mutants (˜85%) had MICs of 16 μg/mL. These colonies had acquired a GC to TA transversion resulting in a Gly 93→Val mutation in FabI, the known protein target of triclosan (FIG. 2B). This mutation is known to produce the greatest triclosan resistance possible via a single step (McMurry L, Oethinger M, Levy S (1998) Nature 394: 531-532). GC to TA transversions are not as readily induced by UV-light as CG to TA transitions. It is, therefore, reasonable that the frequency of UV-induced triclosan-resistant mutants is considerably lower (˜8-fold) than the frequency of UV-induced ciprofloxacin-resistant mutants. The remaining triclosan-resistant mutants (˜15%) had MICs between 2-4 μg/mL. These included three different mutations of Phe 203 in FabI: a TA to CG transition giving serine, a TA to GC transversion giving valine, and a TA to GC transversion giving cystine (FIG. 2B). Similarly, although the untreated and 2.5 ng/mL AZT-treated cultures had much lower frequencies of resistant mutants that made the same-scale analysis unfeasible, the few colonies that emerged were primarily Ser83→Leu mutations on ciprofloxacin and Gly93→Val mutations on triclosan.

Importantly, AZT had the same MX (10 ng/mL) against both the wild-type and the isolated mutant cells (FIG. 2B). AZT is, therefore, not selectively killing the mutant cells versus the wild-type cells to give a false appearance of inhibited mutagenesis. Several rifampicin-resistant mutants had varying sensitivities to AZT. Consequently, the focus was on ciprofloxacin and triclosan selections to investigate the effects of AZT on induced genetic mutations that confer antibacterial resistance.

The largest observable fold-reduction in mutation frequency. In mutagenesis experiments, the fold-induction of mutation is by definition the ratio of the number of induced mutants over the number of spontaneous mutants before outgrowth. Because mutants can be counted only after outgrowth, determining the number of mutants before outgrowth requires a calculation (numbers for 25.8 J/m² of UV treatment and selection on ciprofloxacin are shown in parenthesis): the multiplication of the viable cells per milliliter directly after UV treatment (2.0×10⁷), the median mutation frequency from the selection plate (3.2×10⁻⁷), the volume of cells used to inoculate the overnight (3.0), and a correction factor accounting for the fact that only a percentage of the genuine mutants plated actually form colonies (2.5—only 40% of S83L mutants plated form colonies on 100 ng/mL ciprofloxacin). Using this calculation, 25.8 J/m² of UV light induced about 50 independent GyrA Ser 83→Leu ciprofloxacin-resistant mutants. An untreated culture had on average no GyrA Ser 83 →Leu mutants before outgrowth. Therefore, UV-light induced 50 ciprofloxacin-resistant mutants, and AZT reduced mutation at least 50-fold. Similar calculations were performed for each UV dose and antibiotic selection. 50-fold was the largest observed induction of mutation of the four conditions investigated, and, consequently, it is also the maximum reduction in mutagenesis AZT could effect.

AZT Inhibits UV-induced Lac⁻ to Lac⁺ reversion. The effect of AZT on the UV-induced reversion from Lac⁻ to Lac⁺ in an E. coli strain with an inactivating point mutation in the lacZ gene was investigated. An active β-galactosidase enzyme, the protein product of lacZ, is required for growth when lactose is the only available carbon source. J. H. Miller and co-workers designed a set of six bacterial strains that each require a different one of the six possible base substitutions at the same coding position in the lacZ gene to restore the active site glutamic acid at position 461 (Cupples C, Miller J, (1989) Proc Nat Acad Sci USA, 86: 5345-5349). One strain, CC102, required a GC to AT transition. This strain was chosen because it requires the same transition mutation that generates the GyrA Ser 83 Leu ciprofloxacin-resistant mutant and because it is one of the most frequently reverted by UV light. Similar to the antibiotic selection systems, 5.0 ng/mL AZT, ¼ of the AZT MIC of 20 ng/mL of CC 102, inhibited the UV-induced reversion from Lac⁻ to Lac⁺ (FIG. 2C). Because this mutation is metabolically silent until plated on the lactose minimal media, the reduction in mutation by AZT cannot be ascribed to differences in fitness between wild-type and antibiotic-resistant bacteria.

AZT Inhibits MMS-induced mutagenesis. The effect of AZT on methane methylsulfonate (MMS)-induced mutagenesis was also examined. Unlike UV light, where virtually all mutations are dependent on Pol V, only 70-80% of MMS-induced mutations depend on Pol V (Sledziewska-Gojska E, Janion C, (1989) Mol Gen Genet. 216: 126-31; Grzesiuk E, Janion C, (1994) Mol Gen Genet. 245: 486-492). MMS, although a relatively weak mutagen, effectively induces CG to TA transitions (Nieminuszczy J, Janion C, Grzeiuk E. (2006) Acta Bio Polonica 53: 425-428), making ciprofloxacin an effective selection antibiotic. 2.5 ng/mL AZT reduced the median frequency of MMS-induced ciprofloxacin-resistant mutants about six-fold (FIG. 2D). The effect of AZT again paralleled the effect of the genetic deletion of umuDC in this system, as it did in the UV-induced mutagenesis experiments. In this case, this notably included the failure to completely suppress mutagenesis, which fits nicely with the fact that some 20-30% of MMS-induced mutations do not depend on Pol V.

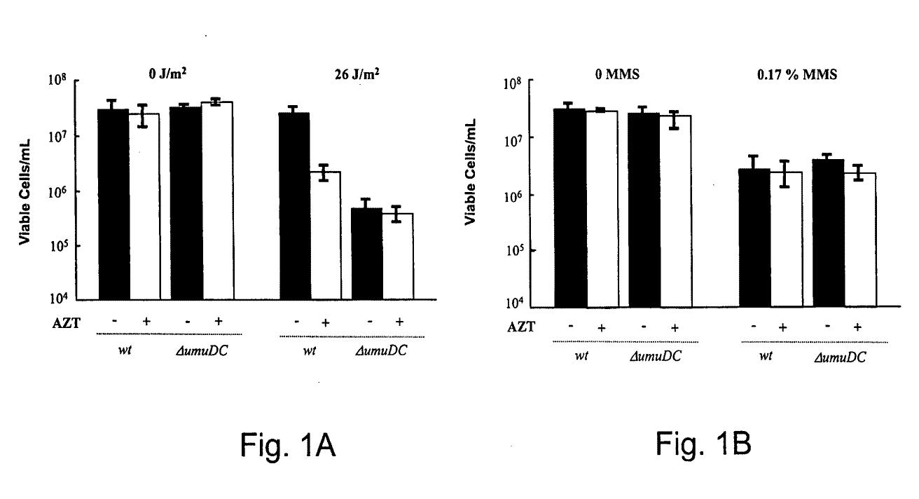

AZT Inhibits Pol V. ΔumuDC E. coli are more sensitive than wild-type E. coli to UV light (Bagg A, Kenyon C J, Walker G C (1981) Proc Natl Acad Sci USA 78:5749-5753) but not to MMS (Nieminuszczy, J, Sikora A, Wrzesinkski M, Janion C, Grzesiuk E (2006) DNA Repair 5: 181-198). If AZT suppresses induced mutation by inhibiting Pol V, then AZT should render wild-type E coli as sensitive to being killed by UV light as ΔumuDC E. coli, but should have no effect on sensitivity to MMS. To test this prediction, both wild-type and ΔumuDC strains were treated as usual with UV or MMS, diluted appropriately, and plated on LB plates or LB plates containing 2.5 ng/mL AZT. FIG. 1 shows that AZT had no effect on the number of viable cells from either strain, if the cultures are either untreated or are MMS treated, although counts were lower for MMS treated cultures. FIG. 1 also shows that ΔumuDC cells were 35-fold more sensitive to UV light than wild-type cells in the absence of AZT, and that the addition of AZT rendered wild type cells 13-fold more sensitive to UV treatment but had no further effects on the survival of the ΔumuDC strain. These results further indicate that AZT mediates its effects through inhibition of Pol V.

Discussion. In wild-type MG1655 K12 E. coli, it was shown that AZT inhibits the UV induction of mutations that confer resistance to two important antibiotics, ciprofloxacin and triclosan (FIG. 2A). E. coli acquire resistance to these two antibiotics via different types of genetic mutations: ciprofloxacin exclusively through a CG to TA transition; and triclosan primarily through a GC to TA transversion. The antibiotic-resistant mutants were not more susceptible to AZT, so the anti-mutagenic effect cannot be attributed to the selective killing of these cells (FIG. 2B). Other chain-terminating nucleoside analogs metabolized by E. coli should have similar effects, and this was confirmed by demonstrating that d4T (6 μg/mL) also inhibited the UV-induced generation of mutations that confer resistance to ciprofloxacin (data not shown). AZT was much more effective, inhibiting the appearance of resistant mutants three orders of magnitude lower in concentration.

To eliminate the possibility that the reduction in mutagenesis was an artifact stemming from phenotype differences between wild-type and antibiotic-resistant bacteria, it was demonstrated that AZT inhibited a UV-induced lactose reversion mutation (FIG. 2C). An active β-galactosidase enzyme is required for growth when the only available carbon source is lactose, but is not expressed and consequently has no effect on fitness in rich media. J. H. Miller designed a series of E. coli strains with inactive β-galactosidase enzymes, each requiring a specific substitution mutation to restore function; one of the strains, CC102, requires a GC to AT transition, the same mutation required for ciprofloxacin-resistance. In a UV mutagenesis experiment, the acquisition of this mutation is metabolically silent in the rich recovery media, where AZT is administered, but essential for growth on lactose minimal selection media. Paralleling the inhibition of the mutations that confer antibiotic-resistance, AZT inhibited the induction of Lac⁺ cells in the CC102 E. coli strain. This result confirms that AZT is inhibiting the induction of mutation, not the outgrowth of mutants due to the different phenotype of the antibiotic-resistant mutant.

MMS-induced mutation was explored to determine if AZT can inhibit-chemically induced, as well as UV-induced mutation. MMS is effective in producing CG to TA transitions, and, therefore, in producing ciprofloxacin-resistant mutants. 2.5 ng/mL AZT reduced the median frequency of MMS-induced ciprofloxacin-resistant mutants about six-fold, identical to the reduction seen with the genetic deletion of umuDC (Table 4). The failure to completely suppress mutagenesis agrees with previously published data indicating only 70-80% of MMS-induced mutations depend on error-prone synthesis.

The striking similarity between the effects of AZT treatment and genetic deletion of Pol V on UV- and chemical-induced mutation strongly suggests that AZT inhibits Pol V. Both completely eliminated UV-induced mutagenesis, and both reduced MMS-induced mutagenesis only 70-80%. Further evidence that AZT inhibits Pol V comes from the sensitivity to UV irradiation from both AZT treatment and Pol V deletion. Specifically, compared to wild-type E. coli after exposure to 25.8 J/m² of UV light, 13-fold fewer AZT (2.5 ng/mL) treated wild-type cells survived, and 35-fold fewer ΔumuDC cells survived. AZT treatment did not affect the viability of ΔumuDC cells after UV treatment. The sensitivity of AZT-treated cells to UV light could have two causes, i) the cells have a decreased tolerance for UV-induced DNA lesions because Pol V is unable to perform TLS or ii) that up-regulated Pol V incorporates AZT, chain-terminates synthesis, and installs toxic DNA damage. AZT treatment, like the deletion of Pol V, does not affect MMS-induced killing. This argues against the second possibility as, if this were the case, AZT-treated cultures should also experience increased sensitivity to MMS. Therefore, both the mutation frequencies and the viabilities after mutagen treatment indicate that AZT inhibits Pol V, mirroring the behavior of ΔumuDC E. coli.

The 13-fold survival loss in AZT-treated cells after UV exposure also shows that the anti-mutagenesis effect is not due exclusively to reduced viability. As described previously, the largest observable fold-reduction in mutation for this UV dose was 50-fold. If the AZT treated cells experienced greater than 50-fold in viability, this would eliminate the observed induced mutagenesis because on average all cultures would have no antibiotic-resistant mutants before outgrowth. Instead, the 13-fold reduction in survival demonstrates that a portion of the reduced mutagenesis is from killing of the most damaged cells because the cell can no longer synthesize past the UV-induced lesion. The remaining mutants are likely no longer generated because Pol V is no longer copying nontemplate DNA and installing mutations. The combined effect then drops the total mutants below 1, and eliminates the appearance of mutation. Because MMS has no sensitivity difference, the reduction in mutagenesis is likely due exclusively the inability of Pol V to copy nontemplate lesions and install mutations.

In conclusion, AZT and the related chain-terminating nucleoside analogs have the unique potential to inhibit error-prone synthesis and corresponding induced mutation in E. coli. Their co-administration with other antibiotics may reduce the amount of resistance emerging to these co-administered antibiotics and increase their lifetimes. Similarly, chain-terminating nucleoside/nucleotide analogues should have the potential to selectively inhibit other error-prone DNA polymerases. These polymerases could include Pol IV in E. coli, ones in other species of bacteria, and ones in higher organisms, including humans.

### Example 2

### Inhibition of Error Prone DNA Polymerases in Human Cells

The present invention also has important ramifications for preventing and treating cancer in human cells, because mutations contribute both to oncogenesis and tumor progression (Fishel R, Lescoe M K, Rao M R, Copeland N G, Jenkins N A, Garber J, Kane M, Kolodner R. Cell, 1993, 75:1027-1038; Hollstein M, Sidransky D, Vogelstein B, Harris C C. Science, 1991, 253:49-53; Mitelman F (1991) Catalog of Chromosome Abberation in Cancer, Wiley Liss, New York.), and to resistance to chemotherapy (Casazza A M, Fairchild C R. Cancer Treat Res, 1996, 87:149-171; Volk E L, Schneider E. Cancer Res, 2003, 63:5538-5543; Tonetti D A, Jordan V C. Anticancer Drugs, 1995, 6:498-507). Ironically, because many chemotherapeutic drugs are mutagens, they may themselves play a role in inducing the mutations that give rise to drug resistance and chemotherapy failure (Loeb L A, Loeb K R, Anderson J P. Proc. Natl. Acad. Sci. USA, 2003, 100:776-781; Greene M H. J. Natl. Cancer Inst., 1992, 84:306-312; Sjoblom T, Parvinen M, Landetie. J. Mutat. Res., 1994, 323:41-45).

The hypothesis that compounds similar to those that prevent mutation in bacteria will also prevent mutation in human cells is an extrapolation of the well-established observation that bacterial mutagens are also human carcinogens. In fact, the gold standard for assessing the carcinogenicity of any compound has long been the Ames test (McCann J, Spingarn N E, Kobori J, Ames B N. Proc. Natl. Acad. Sci. USA, 1975, 72:979-983), which relies on the induction of mutations in the bacterial genome. The Ames test employs a variety of Salmonella enterica serovar Typhimurium mutant strains that cannot grow in the absence of exogenously added histidine, because one of the genes required for the synthesis of this amino acid carries a deactivating mutation (i.e., the encoded protein is not functional due to a specific mutation). The deactivating mutations have been designed to test for different classes of mutagens, such as base substitutions or frameshifts. These histidine auxotrophic Salmonella mutant strains are first exposed to the chemical compound under investigation, and then transferred to minimal medium containing only a small amount of histidine, which is quickly consumed; this initial short period of permissive growth provides time to induce mutations. If the chemical compound is a mutagen (and by inference, a carcinogen), then it has a statistical probability of causing the deactivated histidine biosynthesis gene to be mutated back to a functional gene. Reversion of the inactivating mutation thereby restores the ability of the bacteria to grow in the absence of exogenously added histidine, providing a simple and sensitive phenotype for detection. The Ames test has identified the potential health hazard of a wide variety of ‘high-profile’ carcinogens, including flame retardants (Blum A, Ames B N. Science, 1977, 195:17-23), cigarette smoke (Kier L D, Yamasaki E, Ames B N. Proc. Natl. Acad. Sci. USA, 1974, 71:4159-4163), and hair dyes (Ames B N, Kammen H O, Yamasaki E. Proc. Natl. Acad. Sci. USA, 1975, 72:2423-2427).

In addition, the mechanism by which mutation is induced is similar in bacteria and human cells. For example, for many types of DNA damage the induction of mutation requires the non-essential specialized translesion synthesis (TLS) polymerases Rev1, Polζ, or Polη, encoded by REV1, REV3 and REV7, and RAD30, respectively (Gibbs P E, McGregor W G, Maher V M, Nisson P, Lawrence C W. Proc. Natl. Acad. Sci. USA, 1998, 95:6876-6880; Lawrence C W, Maher V M. Phils. Trans. R Soc. Lond. B. Biol. Sci., 2001, 356:41-46; McNally K, Neal J A, McManus T P, McCormick J J, Maher, V M. DNA Repair, 2008, 7:597-604; Anderson P L, Xu F, Xiao W. Cell Res, 2008, 18:162-173). In yeast, where the process of induced mutation has been extensively studied (L is E T, O'Neill B M, Gil-Lemaignere C, Chin J K, Romesberg F E. DNA Repair, 2008, 7:801-810), these genes are part of the RAD6 genetic epistasis group (Lawrence C W, Das G, Christensen R B. Mol. Gen. Genet., 1985, 200:80-85; Lemontt J F. Genetics, 1971, 68:21-33; Minesinger B K, Jinks-Robertson S. Genetics, 2005, 169:1939-1955), which is responsible for post-replication repair (PRR), a process that converts low molecular weight DNA fragments into higher molecular weight DNA after genome replication is complete (di Caprio L, Cox B S. Mutat Res, 1981, 82:69-85). PRR likely involves the filling in of DNA single-stranded gaps that are created after DNA synthesis is re-initiated downstream of a replication block (Lopes M, Foiani M, Sogo J M. Mol Cell, 2006, 21:15-27; Heller R C, Marians K J. Nature, 2006, 439:557-562; Waters L S, Walker G C. Proc. Natl. Acad. Sci. USA, 2006, 103:8971-8976).

Humans have several additional TLS polymerases that have been implicated in induced mutation, including Polι, Polκ, Polν, and Polρ (Johnson R E, Washington M T, Prakash S, Prakash L. J. Biol. Chem., 200, 275:7447-7450; Kunkel T A, Pavlov Y I, Bebenek K. DNA Repair, 2003, 2:135-149; Arana M E, Takata K, Garcia-Diaz M, Wood R D, Kunkel T A. DNA Repair, 2007, 6:213-223). Poll exhibits a strongly template-dependent fidelity in vitro that has been shown to vary by a factor of up to 100,000 (Frank E G, Woodgate R. J. Biol. Chem., 2007, 282:24689-24696). Polκ excels at the extension of mispaired primer termini (Washington M T, Johnson R E, Prakash L, Prakash S. Proc. Natl. Acad. Sci USA, 2002, 99:1910-1914). Poly appears to preferentially misincorporate dTMP opposite dG (Arana M E, Takata K, Garcia-Diaz M, Wood R D, Kunkel T A. DNA Repair, 2007, 6:213-223). Poll is required to replicate through cis-syn thymine dimmers (Johnson R E, Washington M T, Prakash S, Prakash L. J. Biol. Chem., 200, 275:7447-7450).

The human TLS polymerases appear to be functional homologs of the low fidelity LexA-regulated polymerases PolIV and PolV from E. coli. Specifically, based on homology modeling, PolIV and human Polκ, and PolV and human Polη are orthologs (Lee C H, Chandani S, Loechler E L. J. Mol. Graph. Model., 2006, 25:87-102). Interestingly, like PolIV and PolV, the human TLS polymerases function with lower fidelity and are bereft of exonuclease proofreading activity. Thus, it seems likely that the human TLS polymerases will be subject to inhibition by chain terminator nucleoside analogs.

Unfortunately, no information regarding the inhibition of the human TLS polymerases by chain terminators is available. Thus to demonstrate the feasibility of inhibiting mutation in human cells, and also to broaden the scope of chain terminators demonstrated to inhibit this family of related polymerases, TLS polymerase-mediated synthesis in the presence of different potential chain terminator nucleoside triphosphate analogs was examined. These analogs all bear modified nucleobases and were synthesized in the Romesberg lab (Henry A A, Romesberg F E. Curr Opin Chem Biol, 2003, 7:727-733; Henry A A, Yu C, Romesberg F E. J. Am. Chem. Soc., 2003, 125:9638-9646; Hwang G T, Romesberg F E. Nucleic Acid Res, 2006, 34:2037-2045; Matsuda S, Henry A A, Romesberg F E. J Am Chem Soc, 2006, 128:6369-6375; Matsuda S, Romesberg F E. J. Am. Chem. Soc., 2004, 126:14419-14427; McMinn D L, Ogawa A K, Wu Y, Liu J, Schultz P G, Romesberg F E. J. Am. Chem. Soc., 1999, 121:11585-11586). The particular nucleotide analogs examined were selected because they are efficiently inserted into a growing primer strand by different DNA polymerases, but then distort the nascent primer terminus such that DNA synthesis cannot continue. Thus, in the absence of exonuclease proofreading functions (as with the bacterial and human TLS polymerases), they are expected to act as chain terminators.

To test this hypothesis, DNA synthesis by three human TLS polymerases, Pohη, Polι, and Polκ, in the presence of one of thirty different dNTP analogs was examined (the structure of the nucleobase portion of the dNTP analogs examined, as well as their names, are shown in FIG. 3). The ability of these analogs to inhibit DNA synthesis by Dpo4, a homolog of E. coli PolIV from Sulfolobus solfataricus was also examined. In addition to being a homolog of PolIV, which has shown to be required for mutation in E. coli {Cirz, 2005}, Dpo4 has been demonstrated to behave in a fashion analogous to the human TLS Polη (Boudsocq F, Iwai S, Hanaoka F, Woodgate R. Nucleic Acids Res, 2001, 29:4607-4616).

Primer oligonucleotide, d(TAATACGACTC ACTATAGGGAGA, was 5′-radiolabeled with T4 polynucleotide kinase (New England Biolabs) and [γ-³³/]-ATP (GE Biosciences). Radiolabeled primer was annealed to the template oligonucleotide, d(ATTATGCTGA GTGATATCCCT CTTGCTAGGT TACGGCAGGA TCGC), in reaction buffer by heating to 95° C. followed by slow cooling to room temperature. Assay conditions included 40 nM primer-template and 5.1 to 12.5 nM polymerase. The reaction buffer contained 25 mM Tris-HCl (pH 7.5), 1 mM DTT, 0.1 mg/ml BSA, 10% glycerol, and 5 mM MgCl₂. The reactions were initiated by combining the DNA-enzyme mixture with an equal volume (5 μl) of dNTP stock solution resulting in a final concentration of 200 μM for each natural dNTP and 250 μM of one of the unnatural triphosphates (FIG. 3), incubated at 37° C. for 45 to 60 min (depending on polymerase), and quenched by the addition of 20 μl of loading dye (95% formamide, 20 mM EDTA, and sufficient amounts of bromophenol blue and xylene cyanole). The reaction mixtures were resolved by 15% polyacrylamide gel electrophoresis and the radioactivity was quantified by means of a PhosphorImager using ImageQuant software (Molecular Dynamics). Inhibition was manifest as reduced primer extension (resulting in fewer slower migrating DNA products that run as higher bands on the gel).

The initial screens focused on Polη and Dpo4. DNA synthesis was examined in the presence of the 30 dNTP analogs. After multiple trials, several dNTP analogs emerged as the most potent inhibitors. The data for these, along with a variety that did not show inhibition, are shown in FIG. 4. The most potent inhibitors with Dpo4 are PIM and SoNICS (corresponding to lanes 8 and 13 for Dpo4 in FIG. 4, marked with blue arrows) and with Polη were 2MN, DMN, PIM, SoNICS, and 5SICS (corresponding to lanes 6-8, 13, and 14 for polη in FIG. 4). Polι and Polκ were also screened with all 30 dNTP analogs. Again, inhibition was observed, in the case of Polι, with PIM, SoNICS, MOTP, and MTP (see lanes 8, 13, 17, and 19 in Polι section of FIG. 5) and in the case of Polκ, with 2MN, DMN, PIM, and SoNICS (see lanes 6-8 and 13 in Polκ section of FIG. 5).

The data clearly demonstrate that like E. coli. PolV (and by inference Poly homologs from other bacteria), S. solfataricus Dpo4 (and by inference PolIV homologs form other bacteria) and human Polη, Polι, and Polκ (and by inference other human TLS polymerases), should be subject to inhibition by chain terminator nucleoside analogs, most likely due to their reduced fidelity and inability to exonucleolytically remove the incorporated nucleotides. In turn, because the activity of these TLS polymerases may be required to induce mutations that cause cancer, drive its progression, and/or impart it with chemotherapeutic drug resistance, chain terminator nucleoside analogs should have potent anti-cancer activities. These observations also extend the results to include PolIV homologs from bacteria and nucleoside analogs that act as chain terminators due to modifications in either the sugar moiety (i.e., AZT) or the nucleobase (i.e 2MN, DMN, PIM, SoNICS, MOTP, MTP, or 5SICS).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of inhibiting error prone DNA polymerase mediated mutagenesis in a target cell population in a patient, the method comprising:
selecting a patient on the basis that the patient would benefit from reduced mutagenesis of the population;
administering a nucleoside or nucleotide analog to the patient in an amount sufficient to inhibit DNA synthesis mediated by said error prone DNA polymerase in the population, thereby inhibiting mutagenesis in the population.

2. The method of claim 1, wherein the population is a population of bacteria.

3. The method of claim 2, wherein the population comprises pathogenic bacteria.

4. The method of claim 2, wherein the population comprises gram negative bacteria.

5. The method of claim 2, wherein the bacteria comprise E. coli.

6. The method of claim 1, wherein the population comprises cancer cells.

7. The method of claim 6, wherein the cancer cells comprise tumor cells.

8. The method of claim 1, wherein the error prone DNA polymerase is a Y-family polymerase, a Pol IV polymerase, a Pol V polymerase, a human TLS polymerase, or a DnaE2-type polymerase.

9. The method of claim 1, wherein the error prone DNA polymerase is encoded by umuC, umuD, or both, or a homolog thereof.

10. The method of claim 1, wherein the nucleoside or nucleotide analog is a chain terminating nucleoside or nucleotide analog.

11. The method of claim 1, wherein the nucleoside or nucleotide analog is selected from zidovudine, stavudine, PIM, SoNICS, 2MN, DMN, and 5SICS.

12. The method of claim 1, wherein selecting the patient comprises: determining whether the patient suffers from a chronic or acute infection.

13. The method of claim 1, wherein selecting the patient comprises: determining whether the patient suffers from a chronic disease condition, and selecting the patient based on said chronic condition.

14. The method of claim 13, wherein the chronic condition is selected from the group consisting of old age, cystic fibrosis, and a chronic urinary tract infection.

15. The method of claim 1, wherein identifying the patient comprises:
determining whether the patient is receiving or will receive an anti-cancer medication; and,
determining whether the population is likely to mutate to include cancer cells that are resistant to the anti-cancer medication;
wherein a patient that is receiving or that will receive the anti-cancer medication and that comprises the population is selected for administration of the nucleoside or nucleotide analog.

16. The method of claim 15, wherein the population is determined to be at an increased risk for mutagenesis based upon the type of cancer to be treated, the age of the patient, the immune status of the patient, the disease status of the patient, or exposure of the population of cells to a mutagen.

17. The method of claim 1, wherein identifying the patient comprises:
determining whether the patient is receiving or will receive an antibiotic; and,
determining whether the population is likely to mutate to include a resistant bacteria that is resistant to the antibiotic;
wherein a patient that is receiving or that will receive the antibiotic and that comprises the population likely to mutate to include a resistant bacteria is selected for administration of the nucleoside or nucleotide analog.

18. The method of claim 17, wherein the population is determined to be at an increased risk for mutagenesis, based upon the type of antibiotic to be administered, the type of bacterial infection to be treated, the age of the patient, the disease status of the patient, the immune status of the patient, or exposure of the population to a mutagen.

19. The method of claim 18, wherein the population can develop resistance to the antibiotic through a GC to AT transition mutation, a TA to GC mutation, or a frameshift mutation.

20. The method of claim 18, wherein the mutagen is selected from radiation and exposure to a chemical mutagen.

21. The method of claim 1, wherein the population is a population of bacteria and the patient is administered a dosage of the nucleoside or nucleotide analog that is less than an IC 50 for the nucleoside or nucleotide analog against the bacteria.

22. The method of claim 1, wherein the population is a population of bacteria and the patient is administered a dosage of the nucleoside or nucleotide analog that is less than ½ of an IC 50 for the nucleoside or nucleotide analog against the bacteria.

23. The method of claim 1, wherein the population is a population of bacteria and the patient is administered a dosage of the nucleoside or nucleotide analog that is about ¼ of an IC 50 for the nucleoside or nucleotide analog against the bacteria.

24. The method of claim 1, wherein the population is a population of bacteria and the method includes administering an antibiotic to the patient, and wherein inhibiting mutagenesis of the population inhibits development of resistance to the antibiotic by the bacteria.

25. The method of claim 24, wherein the patient is administered a dosage of the nucleoside or nucleotide analog that is about ½ of an IC 50 for the nucleoside or nucleotide analog against the bacteria.

26. The method of claim 24, wherein the patient is administered a dosage of the nucleoside or nucleotide analog that is about ¼ of an IC 50 for the nucleoside or nucleotide analog against the bacteria.

27. The method of claim 24, wherein the antibiotic is selected from ciprofloxacin and triclosan.

28. The method of claim 1, wherein the population is a population of cancer cells and the method includes administering an anti-cancer chemotherapeutic to the patient, wherein inhibiting mutagenesis of the population inhibits development of resistance to the chemotherapeutic by the cancer cells.

29. The method of claim 28, wherein the patient is administered a dosage of the nucleoside or nucleotide analog that is about ½ of an IC 50 for the nucleoside or nucleotide analog against the cancer cells.

30. The method of claim 28, wherein the patient is administered a dosage of the nucleoside or nucleotide analog that is about ¼ of an IC 50 for the nucleoside or nucleotide analog against the cancer cells.

31. The method of claim 1, wherein the method includes administering more than one nucleoside or nucleotide analog to the patient.

32. A method of screening a set of nucleoside or nucleotide analogs to identify whether one or more members of the set inhibits genomic mutation in a target cell population, the method comprising:
contacting the target cell population with at least one member of the set; and,
determining whether the member inhibits genomic mutation in the target cell population.

33. The method of claim 32, wherein the set comprises a library of at least 10 different nucleoside or nucleotide analogs.

34. The method of claim 32, wherein the set comprises a library of at least 50 different nucleoside or nucleotide analogs.

35. The method of claim 32, wherein the set comprises a library of at least 100 different nucleoside or nucleotide analogs.

36. The method of claim 32, wherein the set comprises a library of more than about 500 different nucleoside or nucleotide analogs.

37. The method of claim 32, wherein the target cell population is a population of bacteria.

38. The method of claim 32, wherein contacting the target cell population with the member comprises incubating the cell population in media that comprises the member.

39. The method of claim 38, wherein the media comprises an antibiotic and determining whether the member inhibits genomic mutation in the target cell population comprises determining a rate at which the population develops antibiotic resistance.

40. The method of claim 32, wherein determining whether the member inhibits genomic mutation in the target cell population comprises measuring a reversion rate for one or more known mutation in one or more marker gene initially present in the population, in the presence of the member.

41. The method of claim 40, wherein the method further comprises determining a dose response curve relating concentration of the member to the reversion rate.

42. A method of screening a set of potential Pol V-specific inhibitor compounds for Pol V-specific inhibitory activity, to identify whether one or more members of the set inhibits genomic mutation in a target cell population, the method comprising:
contacting the target cell population with at least one member of the set;
determining whether the member inhibits genomic mutation in the target cell population; and,
comparing a genomic mutation inhibitory activity of the member in the assay to a genomic mutation activity of a positive control Pol V inhibitor, thereby determining the relative inhibitory activity of the member compared to the positive control.

43. The method of claim 42, wherein the set comprises a library of at least 10 different compounds.

44. The method of claim 42, wherein the set comprises a library of non-nucleotide polymerase inhibitors.

45. The method of claim 42, wherein the target cell population is a population of bacteria.

46. The method of claim 42, wherein contacting the target cell population with the member comprises incubating the cell population in media that comprises the member.

47. The method of claim 46, wherein the media comprises an antibiotic and determining whether the member inhibits genomic mutation in the target cell population comprises determining a rate at which the population develops antibiotic resistance.

48. The method of claim 32, wherein determining whether the member inhibits genomic mutation in the target cell population comprises measuring a reversion rate for one or more known mutation in one or more marker gene initially present in the population, in the presence of the member.

49. The method of claim 40, wherein the method further comprises determining a dose response curve relating concentration of the member to the reversion rate.

50. A composition comprising:
an antibiotic or anti-cancer drug; and,
a nucleoside or nucleotide analog;
wherein the antibiotic or anti cancer drug is present in an amount that provides a therapeutic benefit to a patient suffering from a bacterial infection or cancer, respectively, wherein the nucleoside or nucleotide analog is present in an amount that is insufficient to have therapeutic antiviral, antibiotic or anti cancer activity, but wherein the nucleoside or nucleotide analog is present in an amount that reduces a genomic mutation frequency in cancer or bacterial cells in the patient.

51. The composition of claim 50, wherein the nucleoside or nucleotide analog is present in the composition at a dosage of the nucleoside or nucleotide analog that, over a time of administration of the composition, is less than an IC 50 for the nucleoside or nucleotide analog against a target bacteria or cancer cell.

52. The composition of claim 50, wherein the nucleoside or nucleotide analog is present in the composition at a dosage of the nucleoside or nucleotide analog that, over a time of administration of the composition, is less than ½ of an IC 50 for the nucleoside or nucleotide analog against a target bacteria or cancer cell.

53. The composition of claim 50, wherein the nucleoside or nucleotide analog is present in the composition at a dosage of the nucleoside or nucleotide analog that, over a time of administration of the composition, is less than ¼ of an IC 50 for the nucleoside or nucleotide analog against a target bacteria or cancer cell.

54. (canceled)

55. (canceled)

56. (canceled)

57. (canceled)

58. (canceled)

59. (canceled)


SEQUENCE LISTING (2 sequences)

SEQ ID NO: 1 - DNA, 23 bp, Artificial Sequence
  Synthetic oligonucleotide
taatacgactcactatagggaga

SEQ ID NO: 2 - DNA, 45 bp, Artificial Sequence
  Synthetic oligonucleotide
attatgctgagtgatatccctcttgctaggttacggcaggatcgc